(19) United States
(12) Patent Application Publication
(10) Pub. No.: US 2014/0364334 A1
(43) Pub. Date: Dec. 11, 2014

(54) CONGESTIVE HEART FAILURE BIOMARKERS

(71) Applicant: Nuclea Biotechnologies, Inc., Pittsfield, MA (US)

(72) Inventor: Patrick J. Muraca, Pittsfield, MA (US)

(21) Appl. No.: 14/366,194

(22) PCT Filed: Dec. 20, 2012
(86) PCT No.: PCT/US12/70771

§ 371 Date: Jun. 17, 2014

Related U.S. Application Data
(60) Provisional application No. 61/578,522, filed on Dec. 21, 2011.

(51) Int. Cl. G01N 33/68; C12Q 1/68

(52) U.S. Cl. CPC G01N 33/6893; C12Q 1/6883; C12Q 2600/158; C12Q 2600/118; G01N 2800/325; G01N 2800/56; G01N 2333/58; G01N 2333/91177; G01N 2333/90283; G01N 2333/705
USPC 506/9; 506/18; 506/16

(57) ABSTRACT

The invention provides gene and protein markers for cardiac disease and methods for using them to identify patients at risk for developing heart failure, or patients in an early stage of heart failure whose disease is likely to advance to a later stage. The present invention allows a treatment provider to identify those patients whose disease is most likely to develop and/or advance, and to initiate and/or adjust treatment options for such patients accordingly.

### CROSS REFERENCE TO RELATED APPLICATIONS

This application claims priority to U.S. Provisional Patent Application No. 61/578,522, filed Dec. 21, 2011, entitled Congestive Heart Failure Biomarkers, the contents of which is incorporated herein by reference in its entirety.

### REFERENCE TO SEQUENCE LISTING

The present application is being filed along with a Sequence Listing in electronic format. The Sequence Listing file, entitled 20151025PCT_SEQLST.txt, was created on Dec. 20, 2012 and is 24,382 bytes in size. The information in electronic format of the Sequence Listing is incorporated herein by reference in its entirety.

### FIELD OF THE INVENTION

The invention relates to compositions and methods for determining the presence and stage of congestive heart failure in cardiac patients.

### BACKGROUND OF THE INVENTION

Heart failure (HF), also known as congestive heart failure (CHF), generally is defined as the inability of the heart to supply sufficient blood flow to meet the body's needs. It has various diagnostic criteria, and the term heart failure is often incorrectly used to describe other cardiac-related illnesses, such as myocardial infarction (heart attack) or cardiac arrest.

Common causes of heart failure include myocardial infarction (heart attacks) and other forms of ischemic heart disease, hypertension, valvular heart disease, and cardiomyopathy. McMurray J J, Pfeffer M A (2005), “Heart failure”, Lancet 365 (9474): 1877-89. Heart failure can cause a number of symptoms including shortness of breath (typically worse when lying flat, which is called orthopnea), coughing, chronic venous congestion, ankle swelling, and exercise intolerance. Heart failure is often undiagnosed because of a lack of a universally agreed definition and challenges in definitive diagnosis. Treatment commonly consists of lifestyle measures (such as smoking cessation, light exercise including breathing protocols, decreased salt intake and other dietary changes) and medications, and sometimes devices (pacemaker) or even surgery.

Heart failure is a common, costly, disabling, and potentially deadly condition. In developed countries, around 2% of adults suffer from heart failure, but in those over the age of 65, this increases to 6-10%. Dickstein et al., Eur Heart J. 2008 October; 29(19):2388-442. Mostly as a result of the costs of hospitalization, heart failure is associated with high health expenditures; costs have been estimated to amount to about 2% of the total budget of the National Health Service in the United Kingdom, and more than $35 billion in the United States. Stewart S, et al. (June 2002) “The current cost of heart failure to the National Health Service in the UK”, Eur. J. Heart Fail., 4 (3): 361-71; Rosamond W. et al., (January 2008) “Heart disease and stroke statistics—2008 update: a report from the American Heart Association Statistics Committee and Stroke Statistics Subcommittee”, Circulation 117 (4): e25-146.

Heart failure is associated with significantly reduced physical and mental health, resulting in a markedly decreased quality of life. Juenger J, Schellberg D, Kraemer S, et al. (March 2002) “Health related quality of life in patients with congestive heart failure: comparison with other chronic diseases and relation to functional variables”, Heart 87 (3): 235-41; Hobbs F D, et al., (December 2002) “Impact of heart failure and left ventricular systolic dysfunction on quality of life: a cross-sectional study comparing common chronic cardiac and medical disorders and a representative adult population”, Eur. Heart J. 23 (23):1867-76. With the exception of heart failure caused by reversible conditions, the condition usually worsens with time. Although some people survive many years, progressive disease is associated with an overall annual mortality rate of 10%.

Functional classification for the stages of heart failure generally relies on the New York Heart Association Functional Classification. Criteria Committee, New York Heart Association. Diseases of the heart and blood vessels. Nomenclature and criteria for diagnosis, 6th ed. Boston: Little, Brown and co, 1964; 114. The classes (1-IV) are:

Class I: no limitation is experienced in any activities; there are no symptoms from ordinary activities.

Class II: slight, mild limitation of activity; the patient is comfortable at rest or with mild exertion.

Class III: marked limitation of any activity; the patient is comfortable only at rest.

Class IV: any physical activity brings on discomfort and symptoms occur at rest.

The NYHA score documents severity of symptoms, and can be used to assess response to treatment. While its use is widespread, the NYHA score is not very reproducible and doesn't reliably predict the walking distance or exercise tolerance on formal stress testing. Raphael C, Briscoe C, Davies J, et al. (2007) “Limitations of the New York Heart Association functional classification system and self-reported walking distances in chronic heart failure”, Heart 93(4): 476-82.

In its 2001 guidelines the American College of Cardiology/American Heart Association working group introduced an alternate classification/staging system describing four stages of heart failure:

Stage A: At high risk for developing HF in the future but without structural heart disease or symptoms of HF;

Stage B: Structural heart disease but without signs or symptoms of HF;

Stage C: Structural heart disease but with prior or current symptoms of HF;

Stage D: Refractory HF requiring specialized interventions (hospital-based support, a heart transplant or palliative care).

Hunt S A, Abraham W T, Chin M H, et al. (2005) “ACC/AHA 2005 Guideline Update for the Diagnosis and Management of Chronic Heart Failure in the Adult”, Circulation, 112 (12): e154-235; Jessup M, et al., “2009 Focused Update: ACCF/AHA Guidelines for the Diagnosis and Management of Heart Failure in Adults”, Circulation (2009), 119:1977-2016. The ACC staging system is useful in that Stage A encompasses “pre-heart failure”, a stage at which therapeutic intervention can presumably prevent progression to overt symptoms. ACC stage A does not have a corresponding NYHA class. ACC Stage B would correspond to NYHA Class I. ACC Stage C corresponds to NYHA Classes II and III, while ACC Stage D overlaps with NYHA Class IV.

No system of diagnostic criteria has been agreed upon as the gold standard for heart failure. Commonly used systems are the “Framingham criteria” (derived from the Framingham Heart Study), McKee P A, Castelli W P, McNamara P M, Kannel W B (1971) “The natural history of congestive heart failure: the Framingham study”, N. Engl. J. Med. 285 (26): 1441-6; the “Boston criteria”, Carlson K J, Lee D C, Goroll A H, Leahy M, Johnson R A (1985) “An analysis of physicians' reasons for prescribing long-term digitalis therapy in outpatients” Journal of chronic diseases 38 (9): 733-9; the “Duke criteria”, Harlan W R, Oberman A, Grimm R, Rosati R A (1977) “Chronic congestive heart failure in coronary artery disease: clinical criteria”, Ann. Intern. Med. 86 (2): 133-8; and (in the setting of acute myocardial infarction) the “Killip class” Killip T, Kimball J T (1967) “Treatment of myocardial infarction in a coronary care unit: A two year experience with 250 patients”, Am. J. Cardiol. 20 (4): 457-64.

Other methods used to aid in diagnosing HF include imaging (e.g., echocardiography), chest X-rays, electrophysiology (e.g., an electrocardiogram (ECG/EKG) may be used to identify arrhythmias, ischemic heart disease, right and left ventricular hypertrophy, and presence of conduction delay or abnormalities); and blood tests.

Blood tests routinely performed include electrolytes (sodium, potassium), measures of renal function, liver function tests, thyroid function tests, a complete blood count, and often C-reactive protein if infection is suspected. An elevated B-type natriuretic peptide (BNP) is a specific test indicative of heart failure. Additionally, BNP can be used to differentiate between causes of dyspnea due to heart failure from other causes of dyspnea. If myocardial infarction is suspected, various cardiac markers may be used. BNP is a useful indicator for heart failure and left ventricular systolic dysfunction. Ewald B, Ewald D, Thakkinstian A, Attia J (2008), “Meta-analysis of B type natriuretic peptide and N-terminal pro B natriuretic peptide in the diagnosis of clinical heart failure and population screening for left ventricular systolic dysfunction”, Intern Med J, 38 (2):101-13.

Prognosis in heart failure can be assessed in multiple ways including clinical prediction rules and cardiopulmonary exercise testing. Clinical prediction rules use a composite of clinical factors such as lab tests and blood pressure to estimate prognosis. Among several clinical prediction rules for prognosing acute heart failure, the ‘EFFECT rule’ slightly outperformed other rules in stratifying patients and identifying those at low risk of death during hospitalization or within 30 days. Auble T E, Hsieh M, McCausland J B, Yealy D M (2007) “Comparison of four clinical prediction rules for estimating risk in heart failure”, Annals of emergency medicine, 50(2): 127-35, 135.e1-2.

An important method for assessing prognosis in advanced heart failure patients is cardiopulmonary exercise testing (CPX testing). CPX testing is usually required prior to heart transplantation as an indicator of prognosis. Cardiopulmonary exercise testing involves measurement of exhaled oxygen and carbon dioxide during exercise. The peak oxygen consumption (VO2 max) is used as an indicator of prognosis.

HF is a progresive disease, and the patient's condition will continue to deteriorate if it remains untreated or treatment is insufficient. The amelioration of current symptoms as well as prevention of progression to the next stage often relies upon lifestyle changes, such changes in diet and an increase in physical activity. Accordingly, an early and accurate diagnosis is crucial if the patient's condition is to be effectively treated, and prevented from worsening. However, the subjective nature and lack of precison of current diagnostic methods often result in an inaccurate assessment of the stage and/or severity of disease.

The present invention provides methods and compositions, including gene and protein biomarkers, for the accurate evaluation of the severity and/or stage of heart disease in patients suffering from heart failure.

### SUMMARY OF THE INVENTION

The present invention is based on a study of patients that have been determined to have developed heart failure (HF), or to be at risk for developing heart failure. The invention provides gene and protein markers and methods for using them to identify those patients who are likely to develop HF or whose existing disease is likely to progress or worsen. The present invention allows a treatment provider to stratify patients; that is, to identify presymptomatic patients most likely to develop HF, and those patients that have been diagnosed with HF whose disease is likely to progress to a later stage without treatment, as well as predicting HF progression, including level of failure and prediction of survival. The cardiac markers of the present invention also are useful for distinguishing between the stages of HF, especially in presymptomatic or asymptomatic patients with structural heart disease (HD) (e.g., Stage B). A clinician can use the cardiac markers of the present invention to identify patients that would benefit from a prevention and/or treatment plan in order to prevent or slow the onset of HF, or its progression to a later stage.

In one aspect, the present invention provides gene or protein markers that are indicative of the likelihood that a patient's HF will progress. In one embodiment, the gene and protein HF markers comprise at least one, and preferably a plurality, of genes selected from the group consisting of genes encoding the following proteins: GSTΩ1, SOD2, KCNE2 and BNP. In a preferred embodiment, the present invention provides a set of cardiac markers comprising GSTΩ1 and SOD2. In another preferred embodiment, the set of cardiac markers comprise KCNE2 and BNP. All of these genes/proteins are up-regulated (overexpressed) in the sera of patients who are at risk of developing HF, or whose HF is likely to progress to a more advanced stage, particularly from Stage B to Stage C.

In one aspect, the present invention provides protein markers that are indicative of the likelihood that a presymptomatic patient is at risk of developing HF, or that a diagnosed HF patient's disease is likely to progress to a more advanced stage, specifically, from stage B to stage C. The protein markers comprise proteins that are differentially expressed in individuals at risk of developing HF, or that a diagnosed HF patient's disease is likely to progress to a more advanced stage. In a preferred embodiment, the present invention provides a set of cardiac protein markers comprising GSTΩ1, SOD2, KCNE2 and BNP. In another preferred embodiment, the set of cardiac protein markers comprise GSTΩ1 and SOD2. In another preferred embodiment, the cardiac markers comprise KCNE2 and BNP. In an alternate embodiment, the cardiac markers comprise GSTΩ1 and SOD2, together with one of KCNE2 or BNP. All of these proteins are up-regulated in the sera of patients who at risk of developing HF, or those patients whose diagnosed HF is likely to progress to a later stage.

In one aspect, a method is provided of determining if a presymptomatic patient is likely to develop HF, or if the disease of a patient diagnosed with HF is likely to progress to a more advanced stage. The method comprises obtaining a blood or serum sample from the patient, detecting the presence and amounts of proteins in the sample, and determining whether at least two, and preferably three, and more preferably all four, of the genes or encoded proteins selected from the group consisting of GSTΩ1, SOD2, KCNE2 and BNP are overexpressed from about 2-fold to about 4-fold compared to the levels of these genes/proteins in the sera of normal individuals. In another embodiment, the method comprises determining the levels of the genes or encoded proteins comprising GSTΩ1 and SOD2. In another embodiment, the method comprises determining the levels of the genes or encoded proteins comprising KCNE2 and BNP. In an alternate embodiment, the method comprises determining the levels of the genes or encoded proteins comprising GSTΩ1 and SOD2, together with one of KCNE2 or BNP. All of these genes/proteins are overexpressed compared to their levels in the sera of normal individuals, in the sera of patients who are at risk of developing HF, or those patients whose diagnosed HF are likely to progress to a later stage, in particular, from Stage B to Stage C. From this information, the treatment provider can ascertain whether the patient's HF disease is likely to develop or worsen, and tailor the patient's treatment accordingly.

In some embodiments, the present invention provides a method of predicting whether a patient afflicted with early-stage heart failure will progress to a later stage. This method comprises the steps of obtaining a biologic sample from the subject and determining the expression level of about two, about three or about four biomarkers in the biologic sample, wherein the biomarkers are selected from the group consisting of GSTΩ1, SOD2, KCNE2 and BNP. In some embodiments, the biologic sample may be selected from the group consisting of blood, peripheral blood mononuclear cells (PBMC), isolated blood cells, serum and plasma. In some embodiments, the protein expression level of the biomarkers may be determined by immunoassay methods. In some embodiments, the immunoassay method is an enzyme-linked immunosorbant assay (ELISA) method.

In some embodiments, the present invention provides a kit comprising an agent for detecting the presence or level in a biologic sample of about two, about three or about four biomarkers selected from the group consisting of GSTΩ1, SOD2, KCNE2 and BNP. In some embodiments, the agent in the kit is an antibody or fragment thereof.

The present invention further comprises assays for determining the expression levels of the present cardiac marker genes and/or proteins in a patient's sample, and instructions for using the assay. The assay may be based on detection of nucleic acids (e.g., using nucleic acid probes specific for the nucleic acids of interest) or proteins or peptides (e.g., using antibodies specific for the proteins/peptides of interest). In a preferred embodiment, the assay comprises an immunoassay test in which serum samples are contacted with antibodies specific for the cardiac proteins/peptides identified in the present invention as being indicative of the likelihood that the patient is at risk for developing HF, or that the patients diagnosed HF will progress to a later stage.

In some embodiments, the present invention provides an array comprising, for each of at least two of four genes: GSTΩ1, SOD2, KCNE2 and BNP, one or more polynucleotide probes complementary and hybridizable to an expression product of the gene.

In some embodiments, the present invention provides a method of determining the likelihood that a patient afflicted with Stage B or Stage C heart failure will advance to a later stage of heart failure. This method includes the steps of first determining the level of about two, about three or about four gene transcripts in a biologic sample obtained from said patient corresponding to the biomarkers selected from the group consisting of GSTΩ1, SOD2, KCNE2 and BNP, secondly comparing each of the levels determined according to the first step with the level of each of the same gene transcripts with a biologic sample from a person not afflicted with heart failure and thirdly determining whether the levels of the gene transcripts of the first step correlate with the levels of the transcripts in the second step wherein the determination is indicative of the patient of the first step having a likelihood of advancing to a later stage of heart failure.

In some embodiments, the present invention provides a method of determining the likelihood that a patient afflicted with early-stage heart failure will progress to a later stage comprising the steps of first, obtaining a sample form the patient, secondly, contacting the sample with a panel of antibodies that includes an antibody that binds to about two, about three or about four of the biomarkers selected from the group consisting of GSTΩ1, SOD2, KCNE2 and BNP, wherein each of the at least two, at least three or at least four antibodies binds to a different biomarker within the group and thirdly, assessing the patient's likely prognosis based upon a pattern of binding or lack of binding of the panel to the sample, wherein across a population of patients presenting with heart failure, a higher level of binding of the antibody that binds to each of the biomarkers correlates with a higher likelihood that the patient will advance to a later stage of heart failure.

Practice of the present invention allows the patient and caregiver to make better clinical decisions, e.g., frequency of monitoring, lifestyle modifications, or design of an appropriate therapeutic regimen that may prevent the onset of HF or prevent or slow its advance.

The details of various embodiments of the invention are set forth in the description below. Other features, objects, and advantages of the invention will be apparent from the description and from the claims.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of methods featured in the invention, suitable methods and materials are described below.

### DEFINITIONS

For convenience, the meaning of certain terms and phrases employed in the specification, examples, and appended claims are provided below. The definitions are not meant to be limiting in nature and serve to provide a clearer understanding of certain aspects of the present invention.

The term “heart failure” or “HF”, also known as congestive heart failure (“CHF”), refers to a condition in which the heart can't pump enough blood to supply sufficient blood flow to meet the needs of the body. Heart failure can cause a number of symptoms including shortness of breath, leg swelling, and exercise intolerance, which result from the inability of the heart to keep up with the demands on it and, specifically, failure of the heart to pump blood with normal efficiency. When this occurs, the heart is unable to provide adequate blood flow to other organs such as the brain, liver and kidneys. Heart failure may be due to failure of the right or left or both ventricles. The signs and symptoms depend upon which side of the heart is failing. They can include shortness of breath (dyspnea), asthma due to the heart (cardiac asthma), pooling of blood (stasis) in the general body (systemic) circulation or in the liver's (portal) circulation, swelling (edema), blueness or duskiness (cyanosis), and enlargement (hypertrophy) of the heart.

The term “treating” as used herein, unless otherwise indicated, means reversing, alleviating, inhibiting the progress of, or preventing, either partially or completely, the symptoms of heart failure in a patient with HF. The term “treatment” as used herein, unless otherwise indicated, refers to the act of treating.

The term “clinical management parameter” refers to a metric or variable considered important in the detecting, screening, diagnosing, staging or stratifying patients, or determining the progression of, regression of and/or survival from a disease or condition. Examples of such clinical management parameters include, but are not limited to survival in years, disease related death, responsiveness to treatment, effectiveness of treatment or the likelihood of progression to a later stage of heart failure.

The term “endpoint” means a final stage or occurrence along a path or progression.

The term “HF assessment endpoint” means an endpoint observation or calculation based on the stage, status or occurrence of the symptoms of HF. Examples of endpoints based on cardiac assessments include, but are not limited to, survival, objective response rate (ORR), time to progression (TTP), progression free survival (PFS), and time to treatment failure (TTF).

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions. The term “gene” as used herein includes variants of the genes identified in Table 2.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The terms “gene expression profile” or “GEP” or “gene signature” refer to a group of genes expressed by a particular cell or tissue type wherein presence of the genes or transcriptional products thereof, taken individually (as with a single gene marker) or together or the differential expression of such, is indicative/predictive of a certain condition.

The phrase “single-gene marker” or “single gene marker” refers to a single gene (including all variants of the gene) expressed by a particular cell or tissue type wherein presence of the gene or transcriptional products thereof, taken individually the differential expression of such, is indicative/predictive of a certain condition.

The phrase “gene-protein expression profile “GPEP” as used herein refers to the group of genes and proteins expressed by a particular cell or tissue type wherein presence of the genes and the proteins, taken together or the differential expression of such, is indicative/predictive of a certain condition. GPEPs are comprised of one or more sets of GEPs and PEPs.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

The term “complementary” as it relates to nucleic acids refers to hybridization or base pairing between nucleotides or nucleic acids, such as, for example, between the two strands of a double-stranded DNA molecule or between an oligonucleotide probe and a target are complementary.

As used herein, an “expression product” is a biomolecule, such as a protein or mRNA, which is produced when a gene in an organism is expressed. An expression product may comprise post-translational modifications. The polypeptide of a gene may be encoded by a full length coding sequence or by any portion of the coding sequence.

The terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

The term “amino acid sequence variant” refers to molecules with some differences in their amino acid sequences as compared to a native sequence. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule.

The present invention contemplates several types of compositions, such as antibodies, which are amino acid based including variants and derivatives. These include substitutional, insertional, deletion and covalent variants and derivatives. As such, included within the scope of this invention are polypeptide based molecules containing substitutions, insertions and/or additions, deletions and covalently modifications. For example, sequence tags or amino acids, such as one or more lysines, can be added to the polypeptide sequences of the invention (e.g., at the N-terminal or C-terminal ends). Sequence tags can be used for polypeptide purification or localization. Lysines can be used to increase solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants,” when referring to proteins, are those with one or more amino acids in the native or starting amino acid sequence removed. Ordinarily, deletional variants will have one or more amino acids deleted in a particular region of the molecule.

“Covalent derivatives,” when referring to proteins, include modifications of a native or starting protein with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol. Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that subdomains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH₂)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid.

The term “protein expression” refers to the process by which a nucleic acid sequence undergoes translation such that detectable levels of the amino acid sequence or protein are expressed.

The terms “protein expression profile” or “PEP” or “protein expression signature” refer to a group of proteins expressed by a particular cell or tissue type (e.g., neuron, coronary artery endothelium, or diseased tissue), wherein presence of the proteins taken individually (as with a single protein marker) or together or the differential expression of such proteins, is indicative/predictive of a certain condition.

The phrase “single protein marker” refers to a single protein (including all variants of the protein) expressed by a particular cell or tissue type wherein presence of the protein or translational products of the gene encoding said protein, taken individually the differential expression of such, is indicative/predictive of a certain condition.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about sixteen amino acids.

The terms “array” and “microarray” refer to any type of regular arrangement of objects usually in rows and columns. As it relates to the study of gene and/or protein expression, arrays refer to an arrangement of probes (often oligonucleotide or protein based) or capture agents anchored to a surface which are used to capture or bind to a target of interest. Targets of interest may be genes, products of gene expression, and the like. The type of probe (nucleic acid or protein) represented on the array is dependent on the intended purpose of the array (e.g., to monitor expression of human genes or proteins). The oligonucleotide- or protein-capture agents on a given array may all belong to the same type, category, or group of genes or proteins. Genes or proteins may be considered to be of the same type if they share some common characteristics such as species of origin (e.g., human, mouse, rat); disease state (e.g., cancer); structure or functions (e.g., protein kinases, tumor suppressors); or same biological process (e.g., apoptosis, signal transduction, cell cycle regulation, proliferation, differentiation). For example, one array type may be a “cancer array” in which each of the array oligonucleotide- or protein-capture agents correspond to a gene or protein associated with a cancer. An “epithelial array” may be an array of oligonucleotide- or protein-capture agents corresponding to unique epithelial genes or proteins. Similarly, a “cell cycle array” may be an array type in which the oligonucleotide- or protein-capture agents correspond to unique genes or proteins associated with the cell cycle.

The terms “immunohistochemistry” or as abbreviated “IHC” as used herein refer to the process of detecting antigens (e.g., proteins) in a biologic sample by exploiting the binding properties of antibodies to antigens in said biologic sample.

The term “immunoassay” refers to a test that uses the binding of antibodies to antigens to identify and measure certain substances. Immunoassays often are used to diagnose disease, and test results can provide information about a disease that may help in planning treatment (for example, when estrogen receptors are measured in prostate cancer). An immunoassay takes advantage of the specific binding of an antibody to its antigen. Monoclonal antibodies are often used as they usually bind only to one site of a particular molecule, and therefore provide a more specific and accurate test, which is less easily confused by the presence of other molecules. The antibodies used must have a high affinity for the antigen of interest, because a very high proportion of the antigen must bind to the antibody in order to ensure that the assay has adequate sensitivity.

The term “PCR” or “RT-PCR”, abbreviations for polymerase chain reaction technologies, as used here refer to techniques for the detection or determination of nucleic acid levels, whether synthetic or expressed.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein in diseased tissues or cells versus normal adjacent tissue. For example, a differentially expressed gene may have its expression activated or completely inactivated in normal versus disease conditions, or may be up-regulated (overexpressed) or down-regulated (underexpressed) in a disease condition versus a normal condition. Such a qualitatively regulated gene may exhibit an expression pattern within a given tissue or cell type that is detectable in either control or disease conditions, but is not detectable in both. Stated another way, a gene or protein is differentially expressed when expression of the gene or protein occurs at a higher or lower level in the diseased tissues or cells of a patient relative to the level of its expression in the normal (disease-free) tissues or cells of the patient and/or control tissues or cells.

The term “detectable” refers to an RNA expression pattern which is detectable via the standard techniques of polymerase chain reaction (PCR), reverse transcriptase-(RT) PCR, differential display, and Northern analyses, or any method which is well known to those of skill in the art. Similarly, protein expression patterns may be “detected” via standard techniques such as Western blots.

The term “complementary” as it relates to arrays refers to the topological compatibility or matching together of the interacting surfaces of a probe molecule and its target. The target and its probe can be described as complementary, and furthermore, the contact surface characteristics are complementary to each other.

The term “antibody” means an immunoglobulin, whether natural or partially or wholly synthetically produced. All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE.

The term “antibody fragment” refers to any derivative or portion of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab′)2, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. The preparation of antibodies, whether monoclonal or polyclonal, is know in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

Moreover, antibodies can also be prepared from any region of the peptides of the invention. In addition, if a polypeptide is a receptor protein, antibodies can be developed against an entire receptor or portions of the receptor, for example, an intracellular domain, an extracellular domain, the entire transmembrane domain, specific transmembrane segments, any of the intracellular or extracellular loops, or any portions of these regions. Antibodies can also be developed against specific functional sites, such as the site of ligand binding, or sites that are glycosylated, phosphorylated, myristylated, or amidated, for example.

The term “biomarker” as used herein refers to a substance indicative of a biological state. According to the present invention, biomarkers include the GPEPs, PEPs, GEPs or combinations thereof. Biomarkers according to the present invention also include any compounds or compositions which are used to identify or signal the presence of one or more members of the GPEPs, PEPs, GEPs or combinations thereof disclosed herein. For example, an antibody created to bind to any of the proteins identified as a member of a PEP herein, may be considered useful as a biomarker, although the antibody itself is a secondary indicator.

The term “biological sample” or “biologic sample” refers to a sample obtained from an organism (e.g., a human patient) or from components (e.g., cells) of an organism. The sample may be of any biological tissue, organ, organ system or fluid. The sample may be a “clinical sample” which is a sample derived from a patient. Such samples include, but are not limited to, sputum, blood, blood cells (e.g., white cells), isolated blood cells, peripheral blood mononuclear cells (PBMCs), amniotic fluid, plasma, semen, bone marrow, and tissue or core or fine needle biopsy samples, urine, peritoneal fluid, and pleural fluid, or cells therefrom. Biological samples may also include sections of tissues such as frozen sections taken for histological purposes. A biological sample may also be referred to as a “patient sample.”

The term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition.

The phrase “method of treating” or its equivalent, when applied, for example, to heart disease, refers to a procedure or course of action that is designed to reduce, reverse, eliminate, ameliorate or prevent the progress of heart disease, especially HF, in an individual, or to alleviate the symptoms of heart disease. “A method of treating” HF or another cardiac disorder does not necessarily mean that the HF or other disorder will, in fact, be completely eliminated, that the symptoms of HF or other cardiac disorder will, in fact, be reduced, or that the symptoms of HF or other disorder will, in fact, be alleviated. Often, a method of treating heart disease will be performed even with a low likelihood of success, but which, given the medical history and estimated survival expectancy of an individual, is nevertheless deemed an overall beneficial course of action.

The term “predicting” means a statement or claim that a particular event will occur in the future.

The term “prognosing” means a statement or claim that a particular biologic event will occur in the future.

The terms “progression” and “disease progression” mean the advancement or worsening of a disease or condition from its initial presentation.

The term “therapeutically effective agent” means a composition that will elicit the biological or medical response of a tissue, organ, system, organism, animal or human that is being sought by the researcher, veterinarian, medical doctor or other clinician.

The term “therapeutically effective amount” or “effective amount” means the amount of the subject compound or combination that will elicit the biological or medical response of a tissue, organ, system, organism, animal or human that is being sought by the researcher, veterinarian, medical doctor or other clinician.

By “amplification” is meant production of multiple copies of a target nucleic acid that contains at least a portion of an intended specific target nucleic acid sequence (GSTΩ1, SOD2, KCNE2, BNP, etc). The multiple copies may be referred to as amplicons or amplification products. Preferably, the amplified target contains less than the complete target gene sequence (introns and exons) or an expressed target gene sequence (spliced transcript of exons and flanking untranslated sequences). For example, GSTΩ1-specific amplicons may be produced by amplifying a portion of the GSTΩ1 target polynucleotide by using amplification primers which hybridize to, and initiate polymerization from, internal positions of the GSTΩ1 target polynucleotide. Preferably, the amplified portion contains a detectable target sequence which may be detected using any of a variety of well known methods.

By “primer” or “amplification primer” is meant an oligonucleotide capable of binding to a region of a target nucleic acid or its complement and promoting nucleic acid amplification of the target nucleic acid. In most cases a primer will have a free 3′ end which can be extended by a nucleic acid polymerase. All amplification primers include a base sequence capable of hybridizing via complementary base interactions either directly with at least one strand of the target nucleic acid or with a strand that is complementary to the target sequence. Amplification primers serve as substrates for enzymatic activity that produces a longer nucleic acid product.

A “target-binding sequence” of an amplification primer is the portion that determines target specificity because that portion is capable of annealing to a target nucleic acid strand or its complementary strand. The complementary target sequence to which the target-binding sequence hybridizes is referred to as a primer-binding sequence.

By “detecting” amplification product is meant any of a variety of methods for determining the presence of an amplified nucleic acid, such as, for example, hybridizing a labeled probe to a portion of the amplified product. A labeled probe is an oligonucleotide that specifically binds to another sequence and contains a detectable group which may be, for example, a fluorescent moiety, a chemiluminescent moiety, a radioisotope, biotin, avidin, enzyme, enzyme substrate, or other reactive group.

By “nucleic acid amplification conditions” is meant environmental conditions including salt concentration, temperature, the presence or absence of temperature cycling, the presence of a nucleic acid polymerase, nucleoside triphosphates, and cofactors which are sufficient to permit the production of multiple copies of a target nucleic acid or its complementary strand using a nucleic acid amplification method. Many well-known methods of nucleic acid amplification require thermocycling to alternately denature double-stranded nucleic acids and hybridize primers.

The term “correlate” or “correlation” as used herein refers to a relationship between two or more random variables or observed data values. A correlation may be statistical if, upon analysis by statistical means or tests, the relationship is found to satisfy the threshold of significance of the statistical test used.

As used herein, the term “later stage of HF” refers to a stage of HF that occurs as the condition progresses from a less severe stage to a more severe stage, such as from Stage A to Stage B, Stage C or Stage D, from Stage B to Stage C or Stage D, or from Stage C to Stage D. In some embodiments, the term may be used to refer to either of the two final stages of the condition, Stages C and/or D, in the absence of a reference to a prior stage.

As used herein, the term “subject” or “patient” refers to any organism to which an embodiment of the invention may be applied, e.g., for experimental, diagnostic, prophylactic, and/or therapeutic purposes. Typical subjects include animals (e.g., mammals such as mice, rats, rabbits, non-human primates, and humans) and/or plants.

As used herein, the term “peripheral blood mononuclear cell” or “PBMC” refers to mononuclear cells that circulate in the blood. Such cells include, but are not limited to monocytes, T-cells, B-cells and natural killer cells.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

As used herein, the term “agent” refers to a substance, compound, nucleic acid or antibody utilized in given scenario or for a specified purpose. In some embodiments, an agent may be a “detection agent” or “agent for detecting”, used herein to describe an agent used in the detection of a biological event or level of a substance to be detected (e.g., a nucleic acid, protein and/or biomarker). Such agents may be nucleic acids and/or proteins (including, but not limited to antibodies and/or fragments thereof). In some embodiments, an agent may be a “capture agent” used herein to describe an agent used to bind or otherwise sequester a desired target (e.g., a protein, biomarker and/or nucleic acid). Capture agents may include, but are not limited to nucleic acids and/or proteins. Capture agents that capture a protein may also be termed “protein-specific agents”.

As used herein, the term “hybridization” refers to the bonding of two complementary strands of nucleic acids through bonds between complementary base pairs. In some embodiments, hybridization may occur between complementary strands of DNA. In some embodiments, hybridization may occur between a single nucleic acid strand and a complementary nucleic acid strand. In some embodiments, hybridization may occur between a nucleic acid probe and a nucleic acid target. As used herein, the term “hybridizable” describes the ability of a nucleic acid or nucleic acid probe to hybridize with a given nucleic acid or nucleic acid probe.

### HF Biomarkers

Described herein are compositions and methods for employing cardiac markers for the prognosis or prediction of the likelihood that a subject at risk for HF will develop the disease, or that HF in a subject having the disease will progress to a more advanced stage, especially from Stage B to Stage C.

Prevention of HF in pre-symptomatic individuals, as well as positive treatment outcomes for those who have developed HF, depend on early and accurate detection and classification of the disease. Changes in diet and lifestyle (e.g., increased exercise, losing weight, quitting smoking) may prevent development of HF, and prompt clinical or therapeutic intervention may stop or delay progression of the disease. Most early detections are achieved with the use of physical examinations, CPX testing, or imaging technologies such as CT, MRI and the like. However, these techniques do not provide any guidance as to the likelihood that an asymptomatic patient will develop HF, or that the disease of a patient diagnosed with HF will progress. Consequently, patients at risk for developing HF (stage A or B) or those diagnosed with early-stage HF (stage C) do not always receive the most beneficial treatment as early as possible, resulting in poorer long-term outcome measures. The cardiac markers of the present invention provide the clinician with a prognostic tool capable of providing valuable information that can positively affect management of the disease. According to the present invention, cardiologists can assay a patient's serum for the presence of members of the markers, and can identify with a high degree of accuracy those patients at risk for developing HF, as well of those HF patients whose disease is likely to progress to a later stage. This information, taken together with other available clinical information including imaging data, allows more effective management of the disease.

In a preferred aspect of the invention, the expression levels of certain genes or proteins in a serum sample from a patient is assayed using an immunoassay, in situ hybridization or other assay technique to identify the expression levels of the genes or proteins of the present invention. The gene or protein expression profile comprises at least about two, preferably three, and most preferably all four the genes or proteins selected from the group consisting of: GSTΩ1, SOD2, KCNE2 and BNP. In another embodiment, the present invention provides a set of cardiac markers comprising GSTΩ1 and SOD2. In another embodiment, the set of cardiac markers comprises KCNE2 and BNP. In another embodiment, the present invention provides a set of cardiac markers comprising GSTΩ1 and SOD2, together with one of KCNE2 or BNP. All of these genes/proteins are up-regulated (overexpressed) in the sera of patients who are at risk of developing HF, or whose HF is likely to progress to a more advanced stage. The cardiac markers of the present invention are particularly useful for identifying patients in Stage B and/or early Stage C, and for determining the likelihood that Stage B HF patients will progress to Stage C. Specifically, these markers can be used to predict with a high degree of accuracy whether a patient diagnosed with Stage B HF is likely to advance to Stage C HF.

In one aspect of the invention, the expression of genes or proteins in a serum sample from a patient at risk for, or afflicted with, HF is assayed using immunoassay or in situ hybridization techniques to identify the expression of the genes or proteins consisting of: GSTΩ1, SOD2, KCNE2 and BNP. Alternatively, the genes or proteins assayed comprise GSTΩ1 and SOD2; KCNE2 and BNP; or GSTΩ1 and SOD2, together with one of KCNE2 or BNP. According to the invention, all of these genes/proteins are differentially expressed in patients who are most likely to develop HF, or whose diagnosed HF is likely to advance to a later stage. Specifically, these genes/proteins were found to be up-regulated (over-expressed) in asymptomatic patients likely to develop HF or patients whose diagnosed HF is likely to advance to a later stage. In particular, the expression levels of all of the genes/proteins are up-regulated between about 2-fold to about 4-fold in patients diagnosed with Stage B HF that are highly likely to advance to Stage C compared to the expression levels of these genes/proteins in healthy patients who are free of cardiac disease. As shown by the results in the Examples, overexpression of these genes/proteins occurs in some patients diagnosed with Stage B HF and most patients diagnosed with Stage C HF, whereas the expression levels of these genes in patients with stable Stage B HF is comparable to their expression in healthy patients who are free of cardiac disease. Assays that determine expression levels of these genes can be used by a clinician to determine if a patient in the early stages of HF is likely to worsen, and prescribe a more aggressive course of treatment to improve that patient's cardiac output.

Methods of the present invention comprise (a) obtaining a biological sample (preferably a blood or serum sample) of a patient at risk for or presenting with HF; (b) contacting the sample with nucleic acid probes or antibodies specific for two or more members of the set of cardiac markers comprising GSTΩ1, SOD2, KCNE2 and BNP, and (c) determining whether two or more of the markers are overexpressed compared to the level of these markers in normal patients who are free of HF disease.

The predictive value of the present cardiac markers for determining the stage and/or likelihood of progression of HF increases with the number of the members found to be up-regulated. Preferably, at least about two, more preferably three, and most preferably all four of the genes and/or proteins of the present cardiac marker set are overexpressed. In a preferred embodiment, normal serum samples (e.g., from individuals that do not have HF or other cardiac disease) are assayed simultaneously, using the same reagents and under the same conditions, with the test subject's serum sample. Preferably, expression levels of at least two reference genes/proteins also are measured at the same time and under the same conditions to ensure that the assay is working properly. The assay is deemed to be working properly if the expression levels of the reference genes/proteins are substantially the same (not differentially expressed) in both the patient sample and the control samples.

In a currently preferred embodiment, the present invention comprises assays and methods for determining the likelihood that HF will develop or advance to a later stage in a cardiac patient. In this embodiment, the present method comprises (a) obtaining a biological sample (blood or serum) of a patient at risk for or afflicted with HF; (b) contacting the sample with antibodies specific for the following proteins: GSTΩ1, SOD2, KCNE2 and BNP; or, alternatively, one of the following subsets: or GSTΩ1 and SOD2; KCNE2 and BNP; or GSTΩ1 and SOD2, together with one of KCNE2 or BNP; and (c) determining whether two or more of the proteins are up-regulated (over-expressed) compared to normal (non-cardiac) patients. The predictive value of the protein markers for determining the likelihood that an asymptomatic patient will develop HF, or that a HF patient's disease will advance, increases with the number of these proteins that are found to be overexpressed in accordance with the invention. Preferably, at least about two, more preferably three, and most preferably all four, of the proteins are upregulated in asymptomatic patients likely to develop HF, or in HF patients whose disease is likely advance to a later stage, particularly from Stage B to Stage C.

In another embodiment, the present invention comprises gene markers that are indicative of the likelihood that an asymptomatic patient will develop HF, or that a HF patient's disease will advance to a later stage. In this embodiment, the present method comprises (a) obtaining a biological sample (blood or serum) of a patient at risk for or afflicted with HF; (b) contacting the sample with nucleic acid probes specific for the following genes (e.g., DNA or mRNA): GSTΩ1, SOD2, KCNE2 and BNP; or, alternatively, one of the following subsets: or GSTΩ1 and SOD2; KCNE2 and BNP; or GSTΩ1 and SOD2, together with one of KCNE2 or BNP; and (c) determining whether two or more of the members of the markers are overexpressed compared to normal (non-cardiac) patients. The results described in the Examples show that these genes/proteins are overexpressed in some patients diagnosed with Stage B HF and most patients diagnosed with Stage C HF, but not in patients having stable Stage B HF (e.g., patients whose disease is likely to remain at Stage B) or in normal healthy patients who are free of cardiac disease. The predictive value of the gene markers for determining the likelihood that an asymptomatic patient will develop HF, or that a HF patient's disease will advance, increases with the number of these genes that are found to be overexpressed in accordance with the invention. Preferably, at least about two, more preferably three, and most preferably all four, of the genes are upregulated in asymptomatic patients likely to develop HF, or in HF patients whose disease is likely advance to a later stage.

The biological sample preferably is a sample of the patient's serum. Alternatively, the sample may be blood or plasma. Preferably, expression of at least two reference genes or proteins also is measured simultaneously with the measurement of the genes or proteins in the present GPEPs. Reference genes useful in cardiac assays are described in the literature. See, e.g., Brattelid et al., BMC Mol. Biol., 11:22 (2010).

The present invention further comprises assays for determining the gene and/or protein expression profile in a patient's sample, and instructions for using the assay. The assay may be based on detection of nucleic acids (e.g., using nucleic acid probes specific for the nucleic acids of interest, preferably mRNA) or proteins or peptides (e.g., using nucleic acid probes or antibodies specific for the proteins/peptides of interest). In one embodiment, the assay comprises an immunoassay test in which test and control serum samples are contacted with antibodies specific for the proteins/peptides identified in the present protein marker sets as being indicative of the likelihood that the HF patient's disease will develop or advance. In a preferred embodiment, the immunoassay comprises an enzyme-linked immunosorbant assay (ELISA) in which serum samples, which preferably have been treated to release the proteins from circulating cells, are arrayed in a microtiter plate or other substrate and contacted with antibodies specific for the proteins/peptides identified in the present protein marker sets as being indicative of the likelihood that the HF patient's disease will advance.

Inclusion of any of the biomarker or diagnostic methods described herein as part of treatment and/or monitoring regimens to predict the stage or likelihood of progression of HF provides an advantage over treatment or monitoring regimens that do not include such a biomarker or diagnostic step, in that patient populations can be identified that will derive the most benefit from early intervention which may prevent onset of HF, or prevent or slow the advance of HF. Specifically, symptomatic or presymptomatic cardiac patients who are at risk for developing HF can be identified, and prescribed preventative treatment thereby preventing or slowing the onset of the disease, and those pre-HF or early-stage HF patients who are likely to progress to a later stage can be identified, and prescribed treatment to prevent or slow advancement of the disease. Therefore, use of the cardiac markers of the present invention allows a treatment provider to identify at an earlier stage those patients which will derive the most benefit from early intervention.

The present invention further provides a method for treating a patient at risk for or having HF, comprising the step of determining the likelihood that a patient's disease will develop or advance using one or more of the present cardiac biomarker sets; and a step of administering to the patient an appropriate treatment regimen for HF given the patient's age, gender, or other therapeutically relevant criteria.

Table 2 includes the NCBI Accession No. of at least one variant of each gene. Other variants of these genes and proteins exist, which can be readily ascertained by reference to an appropriate database such as NCBI Entrez (available via the NIH website). Alternate names for the genes and proteins listed also can be determined from the NCBI site. All of the genes and/or proteins listed in Table 2 are up-regulated (overexpressed) in the sera (circulating cells and circulating proteins in sera) of patients who are at risk for developing HF, or whose diagnosed disease is likely to advance to a later stage.

### Clinical Management Parameters

The invention relates to compositions, methods and assays for detecting, screening for, or diagnosing heart failure (HF); staging or stratifying HF patients; and determining the progression of, regression of and/or survival from HF.

In doing so, the present invention provides methods, algorithms and other clinical tools to augment traditional diagnostic, prognostic and/or therapeutic paradigms. Combination approaches using one or more biomarkers in the determination of the value of one or more clinical management parameters also are envisioned. For example methods of this invention that measure GSTΩ1, SOD2, KCNE2 and BNP can provide potentially superior results to diagnostic assays measuring just one of these biomarkers, as illustrated by the data presented herein. This dual biomarker approach, in combination with imaging techniques would provide even further superiority. Any dual, or multiple, biomarker approach (with or without companion imaging) thus reduces the number of patients that are predicted not to benefit from treatment, and thus potentially reduces the number of patients that fail to receive treatment that may extend their lives significantly.

Clinical management parameters addressed by the present invention include survival in years, disease related death, degree of progression, responsiveness to treatment and effectiveness of treatment (e.g., increased cardiac output).

Having found that expression of two or more of GSTΩ1, SOD2, KCNE2 and BNP is a superior predictor of many of the clinical management parameters important to clinicians treating patients having or suspected of having HF, the present invention involves the rapid and accurate identification of these markers in cells and/or serum.

The method generally comprises the following steps: (a) obtaining a biological sample (optimally containing cells or other cell or fluid) from an HF patient; (b) contacting the sample with a detection agent specific for one of the following marker sets: GSTΩ1 and SOD2; KCNE2 and BNP; GSTΩ1 and SOD2, together with one of KCNE2 or BNP; KCNE2 and BNP, together with one of GSTΩ1 and SOD2; or GSTΩ1, SOD2, KCNE2 and BNP; (c) detecting the presence, amount or levels of the markers in (b); and (d) correlating the presence, amount or levels of the markers in order to aid in the prevention, diagnosis or treatment of HF. Step (d) may further include correlating the marker levels with one or more clinical management parameters and/or imaging data. Clinical management parameters may include, for example, stress testing, cardiac echocardiogram, and cardiac enzymes in blood.

The biological sample may be cells or blood, and preferably is serum or plasma containing cells. However, the cells also may be obtained from cell cultures such as in ex vivo or in situ methods.

The detection agent may a nucleic acid probe specific for one or more of GSTΩ1, SOD2, KCNE2 and BNP, or an antibody specific for one or more of GSTΩ1, SOD2, KCNE2 and BNP.

### Probes

The present invention provides novel nucleic acid based probes useful in the detection of the GSTΩ1, SOD2, KCNE2 and/or BNP genes or proteins in a biological sample. To this end, the present invention includes nucleic acid sequences specific for segments of human GSTΩ1, SOD2, KCNE2 and/or BNP genes which are used in methods of detecting GSTΩ1, SOD2, KCNE2 and/or BNP in nucleic acids (e.g., mRNA) prepared from a biological sample. The invention also includes preferred methods that combine nucleic acid sequences for amplifying and detecting GSTΩ1, SOD2, KCNE2 and/or BNP, individually or in combination.

The present invention also includes a method for detecting and quantifying RNA species specific for GSTΩ1, SOD2, KCNE2 and/or BNP. Moreover, detection of these markers individually and in combination are clinically important because HF in individual patients may express one or more of the markers, such that detecting more than one of the markers decreases the potential of false negatives during diagnosis that might otherwise result if the presence of only one marker was tested.

In one embodiment, commercial antibodies may be used to detect expression. Commercial antibodies that are useful for this purpose are available from Abcam (Cambridge, Mass.).

### In Situ Hybridization (ISH) and Fluorescence In Situ Hybridization (FISH)

The present invention provides methods of detecting target nucleic acids via in situ hybridization and fluorescent in situ hybridization using novel probes. The methods of in situ hybridization were first developed in 1969 and many improvements have been made since. The basic technique utilizes hybridization kinetics for RNA and/or DNA via hydrogen bonding. By labeling sequences of DNA or RNA of sufficient length (approximately 50-300 base pairs), selective probes can be made to detect particular sequences of DNA or RNA. The application of these probes to tissue sections allows DNA or RNA to be localized within tissue regions and cell types. Methods of probe design are known to those of skill in the art. Detection of hybridized probe and target may be performed in several ways known in the art. Most prominently is through the use of detection labels attached to the probes. Probes of the present invention may be single or double stranded and may be DNA, RNA, or mixtures of DNA and RNA. They may also constitute any nucleic acid based construct. Labels for the probes of the present invention may be radioactive or non-radioactive and the design and use of such labels is well known in the art.

### Antibodies

In one embodiment, the present invention utilizes antibodies specific for GSTΩ1, SOD2, KCNE2 and/or BNP in an immunoassay assay, such as ELISA or IHC; the ELISA format is preferred, although other formats may be used.

In one embodiment, commercial antibodies are used. In a currently preferred embodiment, rabbit polyclonal antibodies specific for human GSTΩ1, SOD2, KCNE2 and BNP are used (all available from Abcam, Cambridge, Mass.).

### Determination of Gene Expression Profiles

Methods used to identify gene expression profiles indicative of whether a patient's heart condition is likely to progress to a later stage are generally described here and further described in the Examples herein. Other methods for identifying gene and/or protein expression profiles are known; any of these alternative methods also could be used. See, e.g., Chen et al., NEJM, 356(1):11-20 (2007); Lu et al., PLOS Med., 3(12):e467 (2006); Wang et al., J. Clin. Oncol., 2299):1564 (2004); Golub et al., Science, 286:531-537 (1999).

In one method, parallel testing in which, in one track, those genes are identified which are over-/under-expressed as compared to their expression in normal healthy patients (i.e., patients free of heart disease), and/or in patients that experienced different outcomes; in a second track, those genes are identified comprising chromosomal insertions or deletions as compared to the same normal and disease samples. These two tracks of analysis produce two sets of data. The data are analyzed and correlated using an algorithm which identifies the genes of the gene expression profile (i.e., those genes that are differentially expressed in the cells/sera of interest). Positive and negative controls may be employed to normalize the results, including eliminating those genes and proteins that also are differentially expressed in healthy, HF-free patients, and in patients having a different outcome, and confirming that the gene expression profile is unique to the HF patients whose disease is likely to progress to a later stage.

As an initial step, biological samples are acquired from patients at risk for developing HF (Stage A) or presenting with structural hear disease and/or early-stage HF (Stage B), as defined by the ACC/AHA. See, e.g., Hunt et al. (2005), “ACC/AHA 2005 Guideline Update for the Diagnosis and Management of Chronic Heart Failure in the Adult”, Circulation 112 (12): e154-235. The biological samples preferably include blood, serum or plasma samples from each patient. Clinical information associated with each sample, including treatment with cardiac drugs, surgery or other treatment, outcome of the treatments and recurrence or progression of the disease, is recorded in a database. Clinical information also includes information such as age, sex, medical history, treatment history, symptoms, family history, recurrence (yes/no), etc. Samples of blood/sera from normal (undiseased) healthy individuals can be used as positive controls, and serum samples from HF patients whose disease progressed to a later stage may be used as negative controls.

Gene expression profiles (GEPs) are then generated from the biological samples based on total RNA according to well-established methods. Briefly, a typical method involves isolating total RNA from the biological sample, amplifying the RNA, synthesizing cDNA, labeling the cDNA with a detectable label, hybridizing the cDNA with a genomic array, such as the Affymetrix U133A+B GeneChip®, and determining binding of the labeled cDNA with the genomic array by measuring the intensity of the signal from the detectable label bound to the array. See, e.g., the methods described in Lu, et al., Chen, et al. and Golub, et al., supra, and the references cited therein, which are incorporated herein by reference. The resulting expression data are input into a database.

mRNAs in the samples can be analyzed using commercially available or customized probes or oligonucleotide arrays, such as cDNA or oligonucleotide arrays. The use of these arrays allows for the measurement of steady-state mRNA levels of thousands of genes simultaneously, thereby presenting a powerful tool for identifying effects such as the onset, arrest or modulation of uncontrolled cell proliferation. Hybridization and/or binding of the probes on the arrays to the nucleic acids of interest from the cells can be determined by detecting and/or measuring the location and intensity of the signal received from the labeled probe or used to detect a DNA/RNA sequence from the sample that hybridizes to a nucleic acid sequence at a known location on the microarray. The intensity of the signal is proportional to the quantity of cDNA or mRNA present in the sample. Numerous arrays and techniques are available and useful. Methods for determining gene and/or protein expression levels (including in circulating cells in blood or serum) are described, for example, in U.S. Pat. No. 6,271,002; U.S. Pat. No. 6,218,122; U.S. Pat. No. 6,218,114; and U.S. Pat. No. 6,004,755; and in Carlsson et al., Mol. Cell. Proteomics, 10(5):M110:005033 Epub 2011; Inouye et al., Mol. Sys. Biol., 21(6):441 (2010); Wang et al., J. Clin. Oncol., 22(9):1564-1671 (2004); Borovecki et al.; Proc Natl Acad Sci USA, (2005 Aug. 2; Epub 2005 Jul. 25.);102(31):11023-8; and Schena et al., Science, 270:467-470 (1995); all of which are incorporated herein by reference.

The gene analysis aspect may interrogate gene expression as well as insertion/deletion data. As a first step, RNA is isolated from the serum samples and labeled. Parallel processes are run on the sample to develop two sets of data: (1) over-/under-expression of genes based on mRNA levels; and (2) chromosomal insertion/deletion data. These two sets of data are then correlated by means of an algorithm. Over-/under-expression of the genes in each sample are compared to gene expression in the normal (undiseased) samples and other control samples, and a subset of genes that are differentially expressed in the HF patients likely to progress to a later stage is identified. Preferably, levels of up- and down-regulation are distinguished based on fold changes of the intensity measurements of hybridized microarray probes. A difference of about 2.0 fold or greater is preferred for making such distinctions, or a p-value of less than about 0.05. That is, before a gene is said to be differentially expressed in diseased or suspected diseased versus normal cells, the diseased cell is found to yield at least about 2 times greater or less intensity of expression than the normal cells. Generally, the greater the fold difference (or the lower the p-value), the more preferred is the gene for use as a diagnostic or prognostic tool. Genes identified for the gene signatures of the present invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests can identify the genes most significantly differentially expressed between diverse groups of samples. The Student's t-test is an example of a robust statistical test that can be used to find significant differences between two groups. The lower the p-value, the more compelling the evidence that the gene is showing a difference between the different groups. Nevertheless, since microarrays allow measurement of more than one gene at a time, tens of thousands of statistical tests may be run at one time. Because of this, it is unlikely to observe small p-values just by chance, and adjustments using a Sidak correction or similar step as well as a randomization/permutation experiment can be made. A p-value less than about 0.05 by the t-test is evidence that the expression level of the gene is significantly different. More compelling evidence is a p-value less than about 0.05 after the Sidak correction is factored in. For a large number of samples in each group, a p-value less than about 0.05 after the randomization/permutation test is the most compelling evidence of a significant difference.

Another parameter that can be used to select genes that generate a signal that is greater than that of the non-modulated gene or noise is the measurement of absolute signal difference. Preferably, the signal generated by the differentially expressed genes differs by at least about 20% from those of the normal or non-modulated gene (on an absolute basis). It is even more preferred that such genes produce expression patterns that are at least about 30% different than those of normal or non-modulated genes. For smaller subsets of genes evaluated, such as profiles containing less than 30, less than or about 20 or less than or about 10 genes, the expression patterns may be at least about 40% or at least about 50% different than those of normal or non-modulated genes.

Differential expression analyses can be performed using commercially available arrays, for example, Affymetrix U133A+B GeneChip® arrays (Affymetrix, Inc.). These arrays have probe sets for the whole human genome immobilized on the chip, and can be used to determine up- and down-regulation of genes in test samples. Other substrates having affixed thereon human genomic DNA or probes capable of detecting expression products, such as those available from Affymetrix, Agilent Technologies, Inc. or Illumina, Inc. also may be used. Currently preferred gene microarrays for use in the present invention include Affymetrix U133A+B GeneChip® arrays and Agilent Technologies genomic cDNA microarrays. Instruments and reagents for performing gene expression analysis are commercially available. See, e.g., Affymetrix GeneChip® System. The expression data obtained from the analysis then is input into the database.

For chromosomal insertion/deletion analyses, data for the genes of each HF-patient sample as compared to samples from healthy patients is obtained. The insertion/deletion analysis is generated using an array-based comparative genomic hybridization (“CGH”). Array CGH measures copy-number variations at multiple loci simultaneously, providing an important tool for developing diagnostic and therapeutic targets. Microchips for performing array CGH are commercially available, e.g., from Agilent Technologies. The Agilent chip is a chromosomal array which shows the location of genes on the chromosomes and provides additional data for the gene signature. The insertion/deletion data once acquired from this testing is also input into the database.

The analyses are carried out on the same samples from the same patients to generate parallel data. The same chips and sample preparation are used to reduce variability.

The expression of certain genes known as “reference genes” “control genes” or “housekeeping genes” also is determined, preferably at the same time, as a means of ensuring the veracity of the expression profile. Reference genes are genes that are consistently expressed in many cell types in both diseased and healthy patients, and thus are useful to normalize gene expression profiles. See, e.g., Silvia et al., BMC Cancer, 6:200 (2006); Lee et al., Genome Research, 12(2):292-297 (2002); Zhang et al., BMC Mol. Biol., 6:4 (2005). Determining the expression of reference genes in parallel with the genes in the unique gene expression profile provides further assurance that the techniques used for determination of the gene expression profile are working properly. The expression data relating to the reference genes also is input into the database.

### Data Correlation

The differential expression data and the insertion/deletion data in the database may be correlated with the clinical outcomes information associated with each sample also in the database by means of an algorithm to determine a gene expression profile for determining or predicting progression as well as recurrence of disease and/or disease-related presentations. Various algorithms are available which are useful for correlating the data and identifying the predictive gene signatures. For example, algorithms such as those identified in Xu et al., A Smooth Response Surface Algorithm For Constructing A Gene Regulatory Network, Physiol. Genomics 11:11-20 (2002), the entirety of which is incorporated herein by reference, may be used for the practice of the embodiments disclosed herein.

Another method for identifying gene expression profiles is through the use of optimization algorithms such as the mean variance algorithm widely used in establishing stock portfolios. One such method is described in detail in the patent application US Patent Application Publication No. 2003/0194734. Essentially, the method calls for the establishment of a set of inputs expression as measured by intensity) that will optimize the return (signal that is generated) one receives for using it while minimizing the variability of the return. The algorithm described in Irizarry et al., Nucleic Acids Res., 31:e15 (2003) also may be used. One useful algorithm is the JMP Genomics algorithm available from JMP Software.

The process of selecting gene expression profiles also may include the application of heuristic rules. Such rules are formulated based on biology and an understanding of the technology used to produce clinical results, and then are applied to output from the optimization method. For example, the mean variance method of gene signature identification can be applied to microarray data for a number of genes differentially expressed in subjects with HF.

Other heuristic rules can be applied that are not necessarily related to the biology in question. For example, one can apply a rule that only a certain percentage of the portfolio can be represented by a particular gene or group of genes. Commercially available software such as the Wagner software readily accommodates these types of heuristics (Wagner Associates Mean-Variance Optimization Application). This can be useful, for example, when factors other than accuracy and precision have an impact on the desirability of including one or more genes.

As an example, the algorithm may be used for comparing gene expression profiles for various genes (or portfolios) to ascribe prognoses. The expression profiles (whether at the RNA or protein level) of each of the genes comprising the portfolio are fixed in a medium such as a computer readable medium. This can take a number of forms. For example, a table can be established into which the range of signals (e.g., intensity measurements from detection of the amount of antibodies via ELISA or other assay technique) indicative of disease is input. Actual patient data can then be compared to the values in the table to determine whether the patient samples are normal or diseased. In a more sophisticated embodiment, patterns of the expression signals (e.g., fluorescent intensity) are recorded digitally or graphically. The gene expression patterns from the gene portfolios used in conjunction with patient samples are then compared to the expression patterns. Pattern comparison software can then be used to determine whether the patient samples have a pattern indicative of progression of the disease. Of course, these comparisons can also be used to determine whether the patient is not likely to experience disease progression. The expression profiles of the samples are then compared to the profile of a control cell. If the sample expression patterns are consistent with the expression pattern for progression of HF to a later stage, then (in the absence of countervailing medical considerations) the patient is treated as one would treat an HF patient at a later stage. If the sample expression patterns are consistent with the expression pattern from the normal/control cell then the patient is treated as one whose disease is unlikely to worsen.

A method for analyzing the gene signatures of a patient to determine prognosis of HF is through the use of a Cox hazard analysis program. The analysis may be conducted using S-Plus software (commercially available from Insightful Corporation). Using such methods, a gene expression profile is compared to that of a profile that confidently represents disease progression (i.e., expression levels for the combination of genes in the profile is indicative of likelihood of progression). The Cox hazard model with the established threshold is used to compare the similarity of the two profiles (known progression versus patient) and then determines whether the patient profile exceeds the threshold. If it does, then the patient is classified as one who will progress and is accorded treatment such as adjuvant/preventative therapy. If the patient profile does not exceed the threshold then they are classified as a non-progressing patient. Other analytical tools can also be used to answer the same question such as, linear discriminate analysis, logistic regression and neural network approaches. See, e.g., software available from JMP statistical software.

Numerous other well-known methods of pattern recognition are available. The following references provide some examples:

Weighted Voting: Golub, T R., Slonim, D K., Tamaya, P., Huard, C., Gaasenbeek, M., Mesirov, J P., Coller, H., Loh, L., Downing, J R., Caligiuri, M A., Bloomfield, C D., Lander, E S. Molecular classification of cancer: class discovery and class prediction by gene expression monitoring. Science 286:531-537, 1999.

Support Vector Machines: Su, A I., Welsh, J B., Sapinoso, L M., Kern, S G., Dimitrov, P., Lapp, H., Schultz, P G., Powell, S M., Moskaluk, C A., Frierson, H F. Jr., Hampton, G M. Molecular classification of human carcinomas by use of gene expression signatures. Cancer Research 61:7388-93, 2001. Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

K-nearest Neighbors: Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

Correlation Coefficients: van't Veer L J, Dai H, van de Vijver M J, He Y D, Hart A, Mao M, Peters H L, van der Kooy K, Marton M J, Witteveen A T, Schreiber G J, Kerkhoven R M, Roberts C, Linsley P S, Bernards R, Friend S H. Gene expression profiling predicts clinical outcome of breast cancer, Nature. 2002 Jan. 31; 415(6871):530-6.

The gene expression analysis identifies a gene expression profile (GEP) unique to the cancer samples, that is, those genes which are differentially expressed by the cancer cells. This GEP then is validated, for example, using real-time quantitative polymerase chain reaction (RT-qPCR), which may be carried out using commercially available instruments and reagents, such as those available from Applied Biosystems.

### Determination of Protein Expression Profiles

Not all genes expressed by a cell are translated into proteins, therefore, once a GEP has been identified, it may also be desirable to ascertain whether proteins corresponding to some or all of the differentially expressed genes in the GEP also are differentially expressed by the same cells. Therefore, protein expression profiles (PEPs) are generated from the same suspect sera and control sera used to identify the GEPs.

The preferred method for generating PEPs according to the present invention is by immunohistochemistry (IHC) analysis or ELISA assay. In these methods antibodies specific for the proteins in the PEP are used to interrogate tissue/serum samples from individuals of interest. Other methods for identifying PEPs are known, e.g. in situ hybridization (ISH) using protein-specific nucleic acid probes. See, e.g., Hofer et al., Clin. Can. Res., 11(16):5722 (2005); Volm et al., Clin. Exp. Metas., 19(5):385 (2002). Any of these alternative methods also could be used.

For determining the PEPs, samples of suspect blood/serum samples are obtained from patients. These are the same samples used for identifying the GEP. The serum samples as well as the positive and negative control samples are arrayed in microarrays to enable simultaneous analysis. Microarrays consist of substrates, such as microtiter plates, on which up to about 1000 separate serum samples are assembled in array fashion to allow simultaneous immunological analysis.

Arrays are prepared using two serum samples from each patient. Control samples are also included: serum samples from healthy, heart disease-free individuals are included as a negative control, and serum samples from confirmed/diagnosed HF patients may be used as a positive control.

Proteins in the tissue samples may be analyzed by interrogating the arrays using protein-specific agents, such as antibodies or nucleic acid probes, such as oligonucleotides or aptamers. Antibodies are preferred for this purpose due to their specificity and availability. The antibodies may be monoclonal or polyclonal antibodies, antibody fragments, and/or various types of synthetic antibodies, including chimeric antibodies, or fragments thereof. Antibodies are commercially available from a number of sources (e.g., Abcam, Cell Signaling Technology or Santa Cruz Biotechnology), or may be generated using techniques well-known to those skilled in the art. The antibodies typically are equipped with detectable labels, such as enzymes, chromogens or quantum dots, which permit the antibodies to be detected. The antibodies may be conjugated or tagged directly with a detectable label, or indirectly with one member of a binding pair, of which the other member contains a detectable label. Detection systems for use with are described, for example, in the website of Ventana Medical Systems, Inc. Quantum dots are particularly useful as detectable labels. The use of quantum dots is described, for example, in the following references: Jaiswal et al., Nat. Biotechnol., 21:47-51 (2003); Chan et al., Curr. Opin. Biotechnol., 13:40-46 (2002); Chan et al., Science, 281:435-446 (1998). The assay can be automated using commercially available instruments, such as the Benchmark instruments available from Ventana Medical Systems, Inc. ELISA assays may be carried out using instruments and reagents available from BioTek (Winooski, Vt.).

In one embodiment, the arrays are contacted with antibodies specific for the proteins encoded by the genes identified in the gene expression study as being differentially expressed in HF patients whose conditions had progressed to a later stage in order to determine expression of these proteins in each stage of the disease. The antibodies used to interrogate the arrays are selected based on the genes having the highest level of differential expression. See data in Examples.

The results of the ELISA or other immunoassay will show that in individuals whose HF is likely to progress to a later stage, in particular from Stage B to Stage C, the following proteins were up-regulated: GSTΩ1, SOD2, KCNE2 and BNP. These proteins are upregulated in patients that had progressed to a later stage of HF (Stage C or higher) compared with expression of these proteins in the sera from those patients whose disease did not progress.

### Assays

The present invention further comprises methods and assays for determining or predicting whether a patient's pre-symptomatic heart condition is likely to progress to HF, or if a patient diagnosed or afflicted with HF is likely to progress to a later stage of HF. Assays of the present invention are particularly useful for identifying patients in Stage B and/or early Stage C, and for determining the likelihood that Stage B HF patients will progress to Stage C. According to one aspect, a formatted immunoassay can be used for determining if a serum sample exhibits any of the present GEPs, PEPs or GPEPs. In a preferred aspect, a formatted ELISA assay can be used for determining if a serum sample exhibits any of the present GEPs, PEPs or GPEPs. The assays may be formulated into kits that include all or some of the materials needed to conduct the analysis, including reagents (antibodies, detectable labels, etc.) and instructions.

Any of the compositions described herein may be comprised in a kit. In a non-limiting example, reagents for the detection of PEPs, GEPs, or GPEPs are included in a kit. In one embodiment, antibodies to one or more of the expression products of the genes of the GPEPs disclosed herein are included. Antibodies may be included to provide concentrations of from about 0.1 μg/mL to about 500 μg/mL, from about 0.1 μg/mL to about 50 μg/mL or from about 1 μg/mL to about 5 μg/mL or any value within the stated ranges. The kit may further include reagents or instructions for creating or synthesizing further probes, labels or capture agents. It may also include one or more buffers, such as a nuclease buffer, transcription buffer, or a hybridization buffer, compounds for preparing a DNA template, cDNA, primers, probes or label, and components for isolating any of the foregoing. Other kits of the invention may include components for making a nucleic acid or peptide array including all reagents, buffers and the like and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial or similar container. The kits of the present invention also will typically include a means for containing the detection reagents, e.g., nucleic acids or proteins or antibodies, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. The dye may then be resuspended in any suitable solvent, such as DMSO.

Kits may also include components that preserve or maintain the compositions that protect against their degradation. Such kits generally will comprise, in suitable means, distinct containers for each individual reagent or solution.

Certain assay methods of the invention comprises contacting a serum sample from an individual with a group of antibodies specific for some or all of the genes or proteins of a GPEP, and determining the occurrence of up- or down-regulation of these genes or proteins in the sample. The use of TMAs allows numerous samples, including control samples, to be assayed simultaneously.

The method preferably also includes detecting and/or quantitating control or “reference proteins”. Detecting and/or quantitating the reference proteins in the samples normalizes the results and thus provides further assurance that the assay is working properly. In a currently preferred embodiment, antibodies specific for one or more of the following reference proteins are included: beta-actin (ACTB), glyceraldehyde-3-phosphate dehydrogenase (GAPDH), beta glucoronidase (GUSB) as positive controls while negative controls include large ribosomal protein (RPLP0) and/or transferrin receptor (TRFC). Beta actin may be used as the positive control for IHC. Reference genes useful in cardiac assays are described in the literature.

The present invention further comprises a kit containing reagents for conducting an immunoassay analysis of blood/serum/plasma samples or cells from individuals, e.g., patients, including antibodies specific for at least about two of the proteins in a GPEP and for any reference proteins. The antibodies are preferably tagged with means for detecting the binding of the antibodies to the proteins of interest, e.g., detectable labels. Preferred detectable labels include fluorescent compounds or quantum dots; however other types of detectable labels may be used. Detectable labels for antibodies are commercially available, e.g. from Ventana Medical Systems, Inc.

The present invention provides for new assays useful in the diagnosis, prognosis and prediction of HF and the elucidation of clinical management parameters associated with HF. The immunoassays of the present invention utilize the polyclonal or monoclonal antibodies described herein to specifically bind to GSTΩ1, SOD2, KCNE2 and/or BNP in a biological sample. Any type of immunoassay format may be used, including, without limitation, enzyme immunoassays (EIA, ELISA), radioimmunoassay (RIA), fluoroimmunoassay (FIA), chemiluminescent immunoassay (CLIA), counting immunoassay (CIA), immunohistochemistry (IHC), agglutination, nephelometry, turbidimetry or Western Blot. These and other types of immunoassays are well-known and are described in the literature, for example, in Immunochemistry, Van Oss and Van Regenmortel (Eds), CRC Press, 1994; The Immunoassay Handbook, D. Wild (Ed.), Elsevier Ltd., 2005; and the references disclosed therein.

The preferred assay format for the present invention is the enzyme-linked immunosorbent assay (ELISA) format. ELISA is a highly sensitive technique for detecting and measuring antigens or antibodies in a solution in which the solution is run over a surface to which immobilized antibodies specific to the substance have been attached, and if the substance is present, it will bind to the antibody layer, and its presence is verified and visualized with an application of antibodies that have been tagged or labeled so as to permit detection. ELISAs combine the high specificity of antibodies with the high sensitivity of enzyme assays by using antibodies or antigens coupled to an easily assayed enzyme that possesses a high turnover number such as alkaline phosphatase (AP) or horseradish peroxidase (HRP), and are very useful tools both for determining antibody concentrations (antibody titer) in sera as well as for detecting the presence of antigen.

There are many different types of ELISAs; the most common types include “direct ELISA,” “indirect ELISA,” “sandwich ELISA” and cell-based ELISA (C-ELISA). Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized the detection antibody is added, forming a complex with the antigen. The detection antibody can be covalently linked to an enzyme, or can itself be detected by a secondary antibody which is linked to an enzyme through bioconjugation. Between each step the plate typically is washed with a mild detergent solution to remove any proteins or antibodies that are not specifically bound. After the final wash step the plate is developed by adding an enzymatic substrate tagged with a detectable label to produce a visible signal, which indicates the quantity of antigen in the sample.

In a typical microtiter plate sandwich immunoassay, an antibody (“capture antibody”) is adsorbed or immobilized onto a substrate, such as a microtiter plate. Monoclonal antibodies are preferred as capture antibodies due to their greater specificity, but polyclonal antibodies also may be used. When the test sample is added to the plate, the antibody on the plate will bind the target antigen from the sample, and retain it in the plate. When a second antibody (“detection antibody”) or antibody pair is added in the next step, it also binds to the target antigen (already bound to the monoclonal antibody on the plate), thereby forming an antigen ‘sandwich’ between the two different antibodies.

This binding reaction can then be measured by radio-isotopes, as in a radioimmunoassay format (RIA); by enzymes, as in an enzyme immunoassay format (EIA or ELISA); or other detectable label, attached to the detection antibody. The label generates a color signal proportional to the amount of target antigen present in the original sample added to the plate. Depending on the immunoassay format, the degree of color can be detected and measured with the naked eye (as with a home pregnancy test), a scintillation counter (for an RIA), or with a spectrophotometric plate reader (for an EIA or ELISA).

The assay then is carried out according to the following general steps:

Step 1: Capture antibodies are adsorbed onto the well of a plastic microtiter plate (no sample added);

Step 2: A test sample (such as human serum) is added to the well of the plate, under conditions sufficient to permit binding of the target antigen to the capture antibody already bound to the plate, thereby retaining the antigen in the well;

Step 3: Binding of a detection antibody or antibody pair (with enzyme or other detectable moiety attached) to the target antigen (already bound to the capture antibody on the plate), thereby forming an antigen “sandwich” between the two different antibodies. The detectable label on the detection antibodies will generate a color signal proportional to the amount of target antigen present in the original sample added to the plate.

In an alternative embodiment, sometimes referred to as an antigen-down immunoassay, the analyte (rather than an antibody) is coated onto a substrate, such as a microtiter plate, and used to bind antibodies found in a sample. When the sample is added (such as human serum), the antigen on the plate is bound by antibodies (IgE for example) from the sample, which are then retained in the well. A species-specific antibody (anti-human IgE for example) labeled with an enzyme such as horse radish peroxidase (HRP) is added next, which, binds to the antibody bound to the antigen on the plate. The higher the signal, the more antibodies there are in the sample.

In another embodiment, an immunoassay may be structured in a competitive inhibition format. Competitive inhibition assays are often used to measure small analytes because competitive inhibition assays only require the binding of one antibody rather than two as is used in standard ELISA formats. In a sequential competitive inhibition assay, the sample and conjugated analyte are added in steps similar to a sandwich assay, while in a classic competitive inhibition assay, these reagents are incubated together at the same time.

In a typical sequential competitive inhibition assay format, a capture antibody is coated onto a substrate, such as a microtiter plate. When the sample is added, the capture antibody captures free analyte out of the sample. In the next step, a known amount of analyte labeled with a detectable label, such as an enzyme or enzyme substrate, added. The labeled analyte also attempts to bind to the capture antibody adsorbed onto the plate, however, the labeled analyte is inhibited from binding to the capture antibody by the presence of previously bound analyte from the sample. This means that the labeled analyte will not be bound by the monoclonal on the plate if the monoclonal has already bound unlabeled analyte from the sample. The amount of unlabeled analyte in the sample is inversely proportional to the signal generated by the labeled analyte. The lower the signal, the more unlabeled analyte there is in the sample. A standard curve can be constructed using serial dilutions of an unlabeled analyte standard. Subsequent sample values can then be read off the standard curve as is done in the sandwich ELISA formats. The classic competitive inhibition assay format requires the simultaneous addition of labeled (conjugated analyte) and unlabeled analyte (from the sample). Both labeled and unlabeled analyte then compete simultaneously for the binding site on the monoclonal capture antibody on the plate. Like the sequential competitive inhibition format, the colored signal is inversely proportional to the concentration of unlabeled target analyte in the sample. Detection of labeled analyte can be read on a microtiter plate reader.

In addition to microtiter plates, immunoassays are also may be configured as rapid tests, such as a home pregnancy test. Like microtiter plate assays, rapid tests use antibodies to react with antigens and can be developed as sandwich formats, competitive inhibition formats, and antigen-down formats. With a rapid test, the antibody and antigen reagents are bound to porous membranes, which react with positive samples while channeling excess fluids to a non-reactive part of the membrane. Rapid immunoassays commonly come in two configurations: a lateral flow test where the sample is simply placed in a well and the results are read immediately; and a flow through system, which requires placing the sample in a well, washing the well, and then finally adding an analyte-detectable label conjugate and the result is read after a few minutes. One sample is tested per strip or cassette. Rapid tests are faster than microtiter plate assays, require little sample processing, are often cheaper, and generate yes/no answers without using an instrument. However, rapid immunoassays are not as sensitive as plate-based immunoassays, nor can they be used to accurately quantitate an analyte.

The preferred technique for use in the present invention to detect the amount of GSTΩ1, SOD2, KCNE2 and/or BNP in circulating cells is the sandwich ELISA, in which highly specific monoclonal antibodies are used to detect sample antigen. The sandwich ELISA method comprises the following general steps:

1. Prepare a surface to which a known quantity of capture antibody is bound;

2. (Optionally) block any non specific binding sites on the surface;

3. Apply the antigen-containing sample to the surface;

4. Wash the surface, so that unbound antigen is removed;

5. Apply primary (detection) antibodies that bind specifically to the bound antigen;

6. Apply enzyme-linked secondary antibodies which are specific to the primary antibodies;

7. Wash the plate, so that the unbound antibody-enzyme conjugates are removed;

8. Apply a chemical which is converted by the enzyme into a detectable (e.g., color or fluorescent or electrochemical) signal; and

9. Measure the absorbance or fluorescence or electrochemical signal to determine the presence and quantity of antigen.

In an alternate embodiment, the primary antibody (step 5) is linked to an enzyme; in this embodiment, the use of a secondary antibody conjugated to an enzyme (step 6) is not necessary if the primary antibody is conjugated to an enzyme. However, use of a secondary-antibody conjugate avoids the expensive process of creating enzyme-linked antibodies for every antigen one might want to detect. By using an enzyme-linked antibody that binds the Fc region of other antibodies, this same enzyme-linked antibody can be used in a variety of situations. The major advantage of a sandwich ELISA is the ability to use crude or impure samples and still selectively bind any antigen that may be present. Without the first layer of “capture” antibody, any proteins in the sample (including serum proteins) may competitively adsorb to the plate surface, lowering the quantity of antigen immobilized.

In one embodiment of the present invention, a solid phase substrate, such as a microtiter plate or strip, is treated in order to fix or immobilize a capture antibody to the surface of the substrate. The material of the solid phase is not particularly limited as long as it is a material of a usual solid phase used in immunoassays. Examples of such material include polymer materials such as latex, rubber, polyethylene, polypropylene, polystyrene, a styrene-butadiene copolymer, polyvinyl chloride, polyvinyl acetate, polyacrylamide, polymethacrylate, a styrene-methacrylate copolymer, polyglycidyl methacrylate, an acrolein-ethyleneglycol dimethacrylate copolymer, polyvinylidene difluoride (PVDF), and silicone; agarose; gelatin; red blood cells; and inorganic materials such as silica gel, glass, inert alumina, and magnetic substances. These materials may be used singly or in combination of two or more thereof.

The form of the solid phase is not particularly limited insofar as the solid phase is in the form of a usual solid phase used in immunoassays, for example in the form of a microtiter plate, a test tube, beads, particles, and nanoparticles. The particles include magnetic particles, hydrophobic particles such as polystyrene latex, copolymer latex particles having hydrophilic groups such as an amino group and a carboxyl group on the surfaces of the particles, red blood cells and gelatin particles. The solid phase is preferably a microtiter plate or strip, such as those available from Cell Signaling Technology, Inc.

The capture antibody preferably is one or more monoclonal antibodies described herein that specifically bind to at least a portion of one or more of GSTΩ1, SOD2, KCNE2 and/or BNP proteins. Where microtiter plates or strips are used, the capture antibody is immobilized within the wells. Techniques for coating and/or immobilizing proteins to solid phase substrates are known in the art, and can be achieved, for example, by a physical adsorption method, a covalent bonding method, an ionic bonding method, or a combination thereof. See, e.g., W. Luttmann et al., Immunology, Ch. 4.3.1 (pp. 92-94), Elsevier, Inc. (2006) and the references cited therein. For example, when the binding substance is avidin or streptavidin, a solid phase to which biotin was bound can be used to fix avidin or streptavidin to the solid phase. The amounts of the capture antibody, the detection antibody and the solid phase to be used can also be suitably established depending on the antigen to be measured, the antibody to be used, and the type of the solid phase or the like. Protocols for coating microtiter plates with capture antibodies, including tools and methods for calculating the quantity of capture antibody, are described for example, on the websites for Immunochemistry Technologies, LLC (Bloomington, Minn.) and Meso Scale Diagnostics, LLC (Gaithersburg, Md.).

The detection antibody can be any antibody that will bind specifically to GSTΩ1, SOD2, KCNE2 and/or BNP. Such antibodies are commercially available, for example, from Cell Signaling Technologies, Inc., Santa Cruz Biotechnology, Abcam, EMD Biosciences and others. In one embodiment, the detection antibody may be directly conjugated with a detectable label, or an enzyme. If the detection antibody is not conjugated with a detectable label or an enzyme, then a labeled secondary antibody that specifically binds to the detection antibody is included. Such detection antibody “pairs” are commercially available, for example, from Cell Signaling Technologies, Inc.

Techniques for labeling antibodies with detectable labels are well-established in the art. As used herein, the term “detectable label” refers to a composition detectable by spectroscopic, photochemical, biochemical, immunochemical, or chemical means. The detectable label can be selected, e.g., from a group consisting of radioisotopes, fluorescent compounds, chemiluminescent compounds, enzymes, and enzyme co-factors, or any other labels known in the art. See, e.g., Zola, Monoclonal Antibodies: A Manual of Techniques, pp. 147-158 (CRC Press, Inc. 1987). A detectable label can be attached to the subject antibodies and is selected so as to meet the needs of various uses of the method which are often dictated by the availability of assay equipment and compatible immunoassay procedures. Appropriate labels include, without limitation, radionuclides, enzymes (e.g., alkaline phosphatase, horseradish peroxidase, luciferase, or (3-galactosidase), fluorescent moieties or proteins (e.g., fluorescein, rhodamine, phycoerythrin, GFP, or BFP), or luminescent moieties (e.g., Evidot® quantum dots supplied by Evident Technologies, Troy, N.Y., or Qdot™ nanoparticles supplied by the Quantum Dot Corporation, Palo Alto, Calif.).

Preferably, the sandwich immunoassay of the present invention comprises the step of measuring the labeled secondary antibody, which is bound to the detection antibody, after formation of the capture antibody-antigen-detection antibody complex on the solid phase. The method of measuring the labeling substance can be appropriately selected depending on the type of the labeling substance. For example, when the labeling substance is a radioisotope, a method of measuring radioactivity by using a conventionally known apparatus such as a scintillation counter can be used. When the labeling substance is a fluorescent substance, a method of measuring fluorescence by using a conventionally known apparatus such as a luminometer can be used.

When the labeling substance is an enzyme, a method of measuring luminescence or coloration by reacting an enzyme substrate with the enzyme can be used. The substrate that can be used for the enzyme includes a conventionally known luminescent substrate, calorimetric substrate, or the like. When an alkaline phosphatase is used as the enzyme, its substrate includes chemilumigenic substrates such as CDP-start (4-chloro-3-(methoxyspiro(1,2-dioxetane-3,2′-(5′-chloro)tricyclo[3.3.1.1.-sup.3.7]decane)-4-yl)disodium phenylphosphate) and CSPD® (3-(4-methoxyspiro(1,2-dioxetane-3,2-(5′-chloro)tricyclo[3.3.1.1.sup.3.7]-decane)-4-yl)disodium phenylphosphate) and colorimetric substrates such as p-nitrophenyl phosphate, 5-bromo-4-chloro-3-indolyl-phosphoric acid (BCIP), 4-nitro blue tetrazolium chloride (NBT), and iodonitro tetrazolium (INT). These luminescent or calorimetric substrates can be detected by a conventionally known spectrophotometer, luminometer, or the like.

In one embodiment, the detectable labels comprise quantum dots (e.g., Evidot® quantum dots supplied by Evident Technologies, Troy, N.Y., or Qdot™ nanoparticles supplied by the Quantum Dot Corporation, Palo Alto, Calif.). Techniques for labeling proteins, including antibodies, with quantum dots are known. See, e.g., Goldman et al., Phys. Stat. Sol., 229(1): 407-414 (2002); Zdobnova et al., J. Biomed. Opt., 14(2):021004 (2009); Lao et al., JACS, 128(46):14756-14757 (2006); Mattoussi et al., JACS, 122(49):12142-12150 (2000); and Mason et al., Methods in Molecular Biology: NanoBiotechnology Protocols, 303:35-50 (Springer Protocols, 2005). Quantum-dot antibody labeling kits are commercially available, e.g., from Invitrogen (Carlsbad, Calif.) and Millipore (Billerica, Mass.).

The sandwich immunoassay of the present invention may comprise one or more washing steps. By washing, the unreacted reagents can be removed. For example, when the solid phase comprises a strip of microtiter wells, a washing substance or buffer is contacted with the wells after each step. Examples of the washing substance that can be used include 2-[N-morpholino]ethanesulfonate buffer (MES), or phosphate buffered saline (PBS), etc. The pH of the buffer is preferably from about pH 6.0 to about pH 10.0. The buffer may contain a detergent or surfactant, such as Tween 20.

The sandwich immunoassay can be carried out under typical conditions for immunoassays. The typical conditions for immunoassays comprise those conditions under which the pH is about 6.0 to 10.0 and the temperature is about 30 to 45° C. The pH can be regulated with a buffer, such as phosphate buffered saline (PBS), a triethanolamine hydrochloride buffer (TEA), a Tris-HCl buffer or the like. The buffer may contain components used in usual immunoassays, such as a surfactant, a preservative and serum proteins. The time of contacting the respective components in each of the respective steps can be suitably established depending on the antigen to be measured, the antibody to be used, and the type of the solid phase or the like.

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for predicting prognostic outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, and the like.

The methods provided by the present invention may also be automated in whole or in part. The invention is further illustrated by the following non-limiting examples.

### EXAMPLES

### Example 1

### Gene Expression Profile (GEP) Analysis

Gene expression profiles of blood samples were generated for 1068 patients in clinical studies CHF 0001 and CHF 0002. Metrics associated with the two clinical study subsets are shown in Table 1. The setting for both studies was inpatient treatment for heart failure.

Gene expression data from the two studies was obtained via gene array methodology utilizing the Affymetrix HU133A-B GeneChip® whereby blood samples were obtained from patients who had been diagnosed by a cardiologist or internist with either Stage B or Stage C HF. Blood samples from twenty healthy patients (free of cardiac disease) were used as negative controls and were simultaneously processed using the same techniques. The blood samples were subjected to density gradient centrifugation, and the DNA was extracted from the resulting buffy coat fraction using a commercial kit, such as the Qiagen® EZ1 DNA Blood kit and the EZ1 DNA Buffy Coat card.

Gene expression profiles (GEPs) then were generated from the biological samples based on total RNA according to well-established methods (See Affymetrix GeneChip® expression analysis technical manual, Affymetrix, Inc, Santa Clara, Calif.). Briefly, total RNA was isolated from the biological sample, amplified and cDNA synthesized. cDNA was then labeled with a detectable label, hybridized with a the Affymetrix HU133A-B GeneChip® genomic array, and binding of the cDNA to the array was quantified by measuring the intensity of the signal from the detectable cDNA label bound to the array.

TABLE 1
Comparison of two clinical study subsets

| | Study Identifier | Study Identifier |
|---|---|---|
| | (CHF 0001) | (CHF 0002) |
| Heart Failure Diagnosis | Stage B and C | Stage B and C |
| Number of patients: | 536 | 532 |
| Total | | |
| Blood draw | Serum | Plasma |
| Identification of HF severity | 204 | 248 |
| Gene array type | Affymetrix | Affymetrix |
| | HU133A-B | HU133A-B |

*The term “severity” in Table 1 refers to the stage and stage progression of HF.

To develop a predictive GPEP (gene-protein expression profile), 37,452 probe sets were filtered by removing (a) probe sets with low expression over all samples; and (b) probe sets with low variance over all samples. This yielded 13,596 probe sets for subsequent analyses. Normalized log2(intensity) values were centered by subtracting the study-specific mean for each probe set, and rescaled by dividing by the pooled within-study standard deviation for each probe set.

A two-stage model-building approach was used to arrive at the best predictive model:

1. A single probe set analysis was used to search for probe sets that showed a difference between the two studies in the relationship between expression level and disease status, either by logistic regression or linear regression. This analysis yielded 586 probe sets.

2. A fit was analyzed with multi-probe-set predictive models. Here, the pre-selected probe sets from the single-probe-set analyses in step (1) were used as the starting point. Then the initial predictive models to each study were fit separately using a threshold gradient descent (TGD) method for regularized classification. Recursive feature elimination (RFE) was applied to attempt to simplify the models without appreciable loss of predictive accuracy.

The model selection criterion was the mean area under the ROC curve (AUC) from 50 replicates of a 4-fold cross-validation. Then from each RFE model series, here, one per study, the model with maximum difference between the selection criteria for the two studies was selected. The TGD method also was used to build predictive models based on expression of two individual probe sets.

Following the procedures outlined above, Signal-to-Noise ratios (S2N) were generated by comparing expression levels between Stage B and Stage C patients (the whole data set).
S2N was calculated based upon the following formula:

S2N=|x₁−x₂|/(s₁+s₂)
where xᵢ is the mean for trial i and sᵢ is the standard deviation for trial i, i=1,2.

Many microtubule-associated genes were identified with large S2N scores. GSTΩ1 (glutathione-S-transferaseΩ1) had the largest ranking score and relatively wide expression range. Other genes with large signal-to-noise (S2N) scores among those with a range of at least 2.5 for log2(expression intensity) and P-value<0.01 for a t-test of the mean expression difference between Stage B and Stage C HF are shown in Table 2. Gene and Protein Reference Sequence refers to the sequence identifier of the gene from the NCBI database.

TABLE 2
Genes/Proteins having statistically significant signal-to-noise scores

| | | Gene and | Signal | | |
|---|---|---|---|---|---|
| | | Protein | to Noise | | SEQ |
| Gene | | Reference | score | | ID |
| Symbol | Gene Name | Sequences* | (S/N) | P value | NO |
| GSTΩ1 | Glutathione-S- | NM_004832.2 | 0.635 | 0.0004 | 1 |
| | transferase Ω 1 | | | | |
| SOD2 | Superoxide | NM_000636.2 | 0.884 | 0.0004 | 2 |
| | dismutase 2 | | | | |
| KCNE2 | potassium | NM_172201.1 | 0.901 | 0.0002 | 3 |
| | voltage-gated | | | | |
| | channel, | | | | |
| | Isk-related | | | | |
| | family, | | | | |
| | member 2 | | | | |
| BNP | Brain | M31776.1 | 0.935 | 0.0003 | 4 |
| | natriuretic | | | | |
| | peptide | | | | |

*Gene sequence reference sequences have the “NM” prefix.

Table 2 sets forth a 4-gene profile or signature that is indicative of expression differences between patients having Stage B or C HF and normal healthy patients who were free of HF. This 4-gene GEP shows the top four differentially expressed genes in the pooled group of Stage B and C HF patients. All of the genes in the GEP were upregulated 2-fold to 4-fold in the HF patients who progressed to Stage C, compared to their levels in the healthy patients, and in those patients that remained stable in Stage B. The longest isoform of each gene is represented in Table 2; however, it is understood that other variants or isoforms of each gene may exist and that these are included within the embodiment of the gene.

Results of the analysis revealed the genes listed in Table 2 were identified as having the largest S2N scores and a relatively wide expression range.

Additional mRNA and protein sequences for the genes listed in Table 2 include those listed in Table 3.

TABLE 3
mRNA and protein variants

| | | | SEQ |
|---|---|---|---|
| Gene | | | ID |
| Symbol | Gene Name | | NO |
| | | mRNA | |
| | | Reference | |
| | | Sequences | |
| GSTΩ1 | Glutathione-S-transferase Ω 1 | NM_001191003.1 | 5 |
| | | NM_001191002.1 | 6 |
| SOD2 | Superoxide dismutase 2 | NM_001024466.1 | 7 |
| | | NM_001024465.1 | 8 |
| BNP | Brain natriuretic peptide | NM_002521.2 | 9 |
| | | Protein | |
| | | Reference | |
| | | Sequences | |
| GSTΩ1 | Glutathione-S-transferase Ω 1 | NP_001177932.1 | 10 |
| | | NP_004823.1 | 11 |
| | | NP_001177931.1 | 12 |
| SOD2 | Superoxide dismutase 2 | NP_000627.2 | 13 |
| | | NP_001019637.1 | 14 |
| KCNE2 | potassium voltage-gated channel | NP_751951.1 | 15 |
| | Isk-related family, member 2 | | |
| BNP | Brain natriuretic peptide | NP_002512.1 | 16 |

Given these findings, the present invention contemplates the use of at least two, at least 3 or at least 4 of the genes as a gene expression profile, the differential expression of which, either alone or in conjunction with imaging, will serve as a predictor of the likelihood of progression in individuals presenting with Stage B or C HF.

### Example 2

### Identification of GEP Subsets

The results of the analysis also identified two two-gene subsets that are indicative of the likelihood that patents with Stage B or C HF will worsen. These two two-gene GEPs are shown in Tables 4 and 5 respectively.

TABLE 4
Genes having statistically significant signal-to-noise scores (HF 1)

| | | Gene and | Signal to | | |
|---|---|---|---|---|---|
| | | Protein | Noise | | SEQ |
| Gene | | Reference | score | P | ID |
| Symbol | Gene Name | Sequences | (S/N) | value | NO |
| GSTΩ1 | Glutathione-S- | NM_004832.2 | 0.635 | 0.0004 | 1 |
| | transferase Ω 1 | | | | |
| SOD2 | Superoxide | NM_000636.2 | 0.884 | 0.0004 | 2 |
| | dismutase 2 | | | | |

TABLE 5
Genes having statistically significant signal-to-noise scores (HF 2)

| | | Gene and | | | |
|---|---|---|---|---|---|
| | | Protein | Signal to | | SEQ |
| Gene | | Reference | Noise score | P | ID |
| Symbol | Gene Name | Sequences | (S/N) | value | NO |
| KCNE2 | Potassium | NM_172201.1 | 0.901 | 0.0002 | 3 |
| | voltage- | | | | |
| | gated | | | | |
| | channel, | | | | |
| | Isk-related | | | | |
| | family, | | | | |
| | member 2 | | | | |
| BNP | Brain | M31776.1 | 0.935 | 0.0003 | 4 |
| | natriuretic | | | | |
| | peptide | | | | |

The results of the expression analyses using the two 2-gene subsets are shown in Tables 6 and 7. These data illustrate that the two-marker model for both subsets (the presence of increased expression of these genes) predicted the likelihood that HF patients having Stage B or C HF would progress to a later stage with an accuracy of about 80-90% for signature 1 and about 90% for signature 2.

TABLE 6
Two-marker GEP predictive of progression of HF

| | | HF Stage B | | | HF Stage C | | |
|---|---|---|---|---|---|---|---|
| | | | | Detection | | | Detection |
| Model | Subset | R | N | Rate | R | N | Rate |
| GSTΩ1/ | All | 191 | 211 | 0.90 | 159 | 194 | 0.82 |
| SOD2 | patients | | | | | | |
| GSTΩ1 | CHF | 138 | 163 | 0.85 | 182 | 223 | 0.81 |
| SOD2 | CHF | 172 | 194 | 0.87 | 175 | 186 | 0.94 |

TABLE 7
Two-marker GEP predictive of progression of HF

| | | HF Stage B | | | HF Stage C | | |
|---|---|---|---|---|---|---|---|
| | | | | Detection | | | Detection |
| Model | Subset | R | N | Rate | R | N | Rate |
| KCNE2/ | All | 236 | 259 | 0.91 | 175 | 200 | 0.86 |
| BNP | patients | | | | | | |
| KCNE2 | CHF | 182 | 201 | 0.90 | 184 | 204 | 0.90 |
| BNP | CHF | 192 | 227 | 0.85 | 162 | 181 | 0.89 |

In Tables 6 and 7, R=True number of detections, N=Total number of patients in subset, Detection Rate=R/N. The detection rate for each condition for all patients, and for only patients with estimated detection probability was set at an arbitrary threshold of 0.5 based on expression level. The Detection Rate for Stage B means that the model detects Stage B stability, e.g., the probability that the HF patient in Stage B will remain in Stage B. None of the four genes were up-regulated in the Stage B patients whose disease was stable at Stage B, i.e., who did not progress to Stage C. All of these genes were upregulated in some of the Stage B patients and most of the Stage C patients. The Detection Rate for Stage C reflects the rate that patients move from Stage B to C, e.g., probability that the HF patient in Stage B will advance to Stage C.

Consequently, the studies provide two-marker GEPs where the level of expression may be employed as a tool, either alone or in conjunction with other GEPs or imaging techniques, to predict progression of HF to a later stage, in particular, from Stage B to Stage C.

### Example 3

### Gene Expression Profile (GEP) Analysis—Large Studies

Gene expression profiles of serum samples were generated for 2363 patients in clinical studies CHF 0003 and CHF 0004. Metrics associated with the two clinical study subsets are shown in Table 8. The setting for both studies was inpatient treatment for heart failure.

Gene expression data from the two studies was obtained via gene array methodology as described in Example 1 utilizing the Affymetrix HU133A-B GeneChip® whereby serum/plasma samples were obtained from patients who had been diagnosed by a cardiologist or internist with either Stage B (CHF 0003) or Stage C(CHF 0004) HF.

TABLE 8
Comparison of two clinical study subsets

| | | Study | |
|---|---|---|---|
| | Study Identifier | Identifier | |
| | (CHF 0003) | (CHF 0004) | Total Population |
| Heart Failure | Stage B | Stage C | Stage B + C |
| Diagnosis | | | |
| Number of | 1166 | 1197 | 2363 |
| patients: | | | |
| Total | | | |
| Blood draw | Serum, Plasma | Serum, Plasma | Serum, Plasma |
| Gene array type | Affymetrix | Affymetrix | Affymetrix |
| | HU133A-B | HU133A-B | HU133A-B |

A predictive GEP was developed using the two-stage approach described in Example 1. Following the procedures outlined in Example 1, Signal-to-Noise ratios (S2N) were generated by comparing expression levels between Stage B and Stage C patients (the whole data set). Twenty healthy patients (free of cardiac disease) were used as negative controls.

The results showing the mean expression difference between Stage B and Stage C HF are shown in Tables 9 and 10.

TABLE 9
Two-marker GEP predictive of progression of HF

| | | Stage B | | | Stage C | | |
|---|---|---|---|---|---|---|---|
| | | | | Detection | | | Detection |
| Model | Subset | R | N | Rate | R | N | Rate |
| None | All | — | 1166 | — | — | 1197 | — |
| | patients | | | | | | |
| GSTΩ1 | CHF | 974 | 1166 | 0.83 | 983 | 1197 | 0.82 |
| SOD2 | CHF | 962 | 1166 | 0.82 | 977 | 1197 | 0.81 |

TABLE 10
Two-marker GEP predictive of progression of HF

| | | Stage B | | | Stage C | | |
|---|---|---|---|---|---|---|---|
| | | | | Detection | | | Detection |
| Model | Subset | R | N | Rate | R | N | Rate |
| None | All | — | 1166 | — | — | 1197 | — |
| | patients | | | | | | |
| KCNE2 | CHF | 958 | 1166 | 0.82 | 963 | 1197 | 0.80 |
| BNP | CHF | 989 | 1166 | 0.85 | 969 | 1197 | 0.81 |

In Tables 9 and 10, R=True number of detections, N=Total number of patients in subset, Detection Rate=R/N. The detection rate for each condition for all patients, and for only patients with estimated detection probability was set at an arbitrary threshold of 0.5 based on expression level. The Detection Rate for Stage B reflects Stage B stability, e.g., the probability that the HF patient in Stage B will remain in Stage B. None of the four genes in Tables 9 and 10 are overexpressed in stable Stage B patients, whereas these genes are overexpressed in some of the Stage B patients and most of the Stage C patients. The Detection Rate for Stage C reflects the probability that the HF patient in Stage B will advance to Stage C.

The results in Tables 9 and 10 show that the detection rates of expression of these markers patients diagnosed with Stage C HF; these results indicate that Stage B HF patients that overexpress these markers are likely to progress to Stage C. Accordingly, the studies provide two-marker GEPs where the level of expression may be employed as a tool, either alone or in conjunction with other GEPs or imaging techniques, to predict progression of HF to a later stage.

The present invention contemplates the use of at least two, at least 3 or at least 4 of the genes as a gene expression profile, the differential expression of which, either alone or in conjunction with imaging, will serve as a predictor of the likelihood of progression in individuals presenting with Stage B or C HF.

1. A method of predicting whether a patient afflicted with early-stage heart failure will progress to a later stage comprising:
(a) obtaining a biologic sample from the subject; and
(b) determining the expression level of about two, about three or about four biomarkers in said biologic sample, wherein the biomarkers are selected from the group consisting of GSTΩ1, SOD2, KCNE2 and BNP.

2. The method of claim 1 wherein the biologic sample obtained is selected from the group consisting of blood, peripheral blood mononuclear cells (PBMC), isolated blood cells, serum and plasma.

3. The method of claim 1 wherein the expression level determined is of the biomarker protein by immunoassay methods.

4. The method of claim 3 wherein the immunoassay method is an enzyme-linked immunosorbant assay (ELISA) method.

5. The method of claim 1 wherein the expression level of about two biomarkers is determined.

6. A kit comprising an agent for detecting the presence or level in a biologic sample of at about two, about three or about four biomarkers selected from the group consisting of GSTΩ1, SOD2, KCNE2 and BNP.

7. The kit of claim 6, wherein the agent is an antibody or a fragment thereof.

8. An array comprising, for each of at least two of four genes: GSTΩ1, SOD2, KCNE2 and BNP, one or more polynucleotide probes complementary and hybridizable to an expression product of the gene.

9. A method of determining the likelihood that a patient afflicted with Stage B or Stage C heart failure will advance to a later stage of heart failure, comprising:
(a) determining the level of about two, about three or about four gene transcripts in a biologic sample obtained from said patient corresponding to the biomarkers selected from the group consisting of GSTΩ1, SOD2, KCNE2 and BNP;
(b) comparing each of the levels determined according to step (a) with the level of each of the same gene transcripts with a biologic sample from a person not afflicted with heart failure; and
(c) determining whether the levels of said gene transcripts of step (a) correlate with the levels of said transcripts in step (b) wherein said determination is indicative of said patient of step (a) having a likelihood of advancing to a later stage of heart failure.

10. A method of determining the likelihood that a patient afflicted with early-stage heart failure will progress to a later stage comprising:
(a) obtaining a sample from the patient;
(b) contacting the sample with a panel of antibodies that includes an antibody that binds to about two, about three or about four of the biomarkers selected from the group consisting of GSTΩ1, SOD2, KCNE2 and BNP, wherein each of the at least two, at least three or at least four antibodies binds to a different biomarker within the group; and
(c) assessing the patient's likely prognosis based upon a pattern of binding or lack of binding of the panel to the sample, wherein across a population of patients presenting with heart failure, a higher level of binding of the antibody that binds to each of the biomarkers correlates with a higher likelihood that the patient will advance to a later stage of heart failure.


SEQUENCE LISTING (16 sequences)

SEQ ID NO: 1 - DNA, 1016 bp, Homo sapiens
ccttggccagcgagatgctttgacacagccccttaagatgttttaagggctatcccataaagccgggaaggcacaacgggtggagctggcggccgccgggggcaggcacttttgagctaaggaggaagcgggggagggaaggagggcgggaaggacgcgccacctacttcctgaatcccctgcaaaccccagaggagctcggcctgcgctgcgccacgatgtccggggagtcagccaggagcttggggaagggaagcgcgcccccggggccggtcccggagggctcgatccgcatctacagcatgaggttctgcccgtttgctgagaggacgcgtctagtcctgaaggccaagggaatcaggcatgaagtcatcaatatcaacctgaaaaataagcctgagtggttctttaagaaaaatccctttggtctggtgccagttctggaaaacagtcagggtcagctgatctacgagtctgccatcacctgtgagtacctggatgaagcatacccagggaagaagctgttgccggatgacccctatgagaaagcttgccagaagatgatcttagagttgttttctaaggtgccatccttggtaggaagctttattagaagccaaaataaagaagactatgctggcctaaaagaagaatttcgtaaagaatttaccaagctagaggaggttctgactaataagaagacgaccttctttggtggcaattctatctctatgattgattacctcatctggccctggtttgaacggctggaagcaatgaagttaaatgagtgtgtagaccacactccaaaactgaaactgtggatggcagccatgaaggaagatcccacagtctcagccctgcttactagtgagaaagactggcaaggtttcctagagctctacttacagaacagccctgaggcctgtgactatgggctctgaagggggcaggagtcagcaataaagctatgtctgatattttccttcactaatatgaataaaaaaaaaaaaaaa

SEQ ID NO: 2 - DNA, 1593 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 3 - DNA, 809 bp, Homo sapiens
gtaaggtgaaggtgcccagcaggctgaggcttgtgtgcaacccagaagagagctcgctaacgccagcaagaaggttcagaacagcctggctttggaaaggaatttcatcctgcccacacactgcatagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctctgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaatttgaagaaaaaa

SEQ ID NO: 4 - DNA, 1922 bp, Homo sapiens
ctgtgagatcaccccgtgctcccagcgctcacgtcggtcctcggaaagccggggtcctccctgccttttccagcaacggtggggtggggaggcaggaagaaagcgccaacctaggaccccggagatttgcagcaaaggaagaagcgggagacgggcacttgtctgtgtctccagcgcgttcctgccccccgccgacccggcccatttctatacaaggtcgctctgcccggtctccacctcccacgtgcaggccgcggaggggctcattcccgggccctgatctcagaggcccggaatgtggctgataaatcagagactagacctgcatggcaggcaggcccgacactcagctccaggataaaaggccacggtgtcccgaggagccaggaggagcaccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggtgagagcggagggcagctcagggggattggacagcagcaatgaaagggtcctcacctgctgtcccaagaggccctcatctttcctttggaattagtgataaaggaatcagaaaatggagagactgggtgccctgaccctgtacccaaggcagtcggttcacttgggtgccatgaagggctggtgagccaggggtgggtccctgaggcttggacgcccccattcattgcaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaaggtaagcaccccctgccaccccggccgccttcccccattccagtgtgtgacactgttagagtcactttggggtttgttgtctctgggaaccacactctttgagaaaaggtcacctggacatcgcttcctcttgttaacagccttcagggccaaggggtgcctttgtggaattagtaaatgtgggcttatttcattaccatgcccacaataccttctccccacctcctacttcttatcaaaggggcagaatctcctttgggggtctgtttatcatttggcagccccccagtggtgcagaaagagaaccaaacatttcctcctggtttcctctaaactgtctatagtctcaaaggcagagagcaggatcaccagagcaatgataatccccaatttacagatgaggaaactgaggctcagagagttgcattaagcctcaaacgtctgatgactaacagggtggtgggtggcacacgatgaggtaagctcagcccctgcctccatctcccaccctaaccatcatcaccctctctctttccctgacagtgctgaggcggcattaagaggaagtcctggctgcagacacctgcttctgattccacaaggggctttttcctcaaccctgtggccgcctttgaagtgactcattttttttaatgtatttatgtatttatttgattgttttatataagatggtttcttacctttgagcacaaaatttccacggtgaaataaagtcaacattataagctttatcttttgaaactgatttgtcttggcgcattaaaaataatccctcatttcaaagaa

SEQ ID NO: 5 - DNA, 1070 bp, Homo sapiens
aaggatgactcctagacctgtctttgtcccatacttgggaagataagctgttagtttacattgccaagatgaaattcaactgatctgttttagagtaaagatcttgcgggccaacaaggaatttccttggggaggtgtgtggcctttgatctttgtagctatttagcaacaaaatgggaggcagtgttgcctgactcagtttccaagcttgactcttccctttggcatagtgagtttggagtcaagagatttttattttcccttcacaagctgaacaggagcctttggagagaacgctctggaccggaagcgcgcccccggggccggtcccggagggctcgatccgcatctacagcatgaggttctgcccgtttgctgagaggacgcgtctagtcctgaaggccaagggaatcaggcatgaagtcatcaatatcaacctgaaaaataagcctgagtggttctttaagaaaaatccctttggtctggtgccagttctggaaaacagtcagggtcagctgatctacgagtctgccatcacctgtgagtacctggatgaagcatacccagggaagaagctgttgccggatgacccctatgagaaagcttgccagaagatgatcttagagttgttttctaaggtgccatccttggtaggaagctttattagaagccaaaataaagaagactatgctggcctaaaagaagaatttcgtaaagaatttaccaagctagaggaggttctgactaataagaagacgaccttctttggtggcaattctatctctatgattgattacctcatctggccctggtttgaacggctggaagcaatgaagttaaatgagtgtgtagaccacactccaaaactgaaactgtggatggcagccatgaaggaagatcccacagtctcagccctgcttactagtgagaaagactggcaaggtttcctagagctctacttacagaacagccctgaggcctgtgactatgggctctgaagggggcaggagtcagcaataaagctatgtctgatattttccttcactaatatgaataaaaaaaaaaaaaaa

SEQ ID NO: 6 - DNA, 918 bp, Homo sapiens
cccttggccagcgagatgctttgacacagccccttaagatgttttaagggctatcccataaagccgggaaggcacaacgggtggagctggcggccgccgggggcaggcacttttgagctaaggaggaagcgggggagggaaggagggcgggaaggacgcgccacctacttcctgaatcccctgcaaaccccagaggagctcggcctgcgctgcgccacgatgtccggggagtcagccaggagcttggggaagggaagcgcgcccccggggccggtcccggagggctcgatccgcatctacagcatgaggttctgcccgtttgctgagaggacgcgtctagtcctgaaggccaagggaatcaggcatgaagtcatcaatatcaacctgaaaaataagcctgagtggttctttaagaaaaatccctttggtctggtgccagttctggaaaacagtcagggtcagctgatctacgagtctgccatcacctgtgagtacctggatgaagcatacccagggaagaagctgttgccggatgacccctatgagaaagcttgccagaagatgatcttagagttgttttctaaggttctgactaataagaagacgaccttctttggtggcaattctatctctatgattgattacctcatctggccctggtttgaacggctggaagcaatgaagttaaatgagtgtgtagaccacactccaaaactgaaactgtggatggcagccatgaaggaagatcccacagtctcagccctgcttactagtgagaaagactggcaaggtttcctagagctctacttacagaacagccctgaggcctgtgactatgggctctgaagggggcaggagtcagcaataaagctatgtctgatattttccttcactaatatgaataaaaaaaaaaaaaaa

SEQ ID NO: 7 - DNA, 918 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgatcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaaaaaaaaaaaa

SEQ ID NO: 8 - DNA, 1035 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgatcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaaaaaaaaaaaa

SEQ ID NO: 9 - DNA, 708 bp, Homo sapiens
ccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaagaggaagtcctggctgcagacacctgcttctgattccacaaggggctttttcctcaaccctgtggccgcctttgaagtgactcatttttttaatgtatttatgtatttatttgattgttttatataagatggtttcttacctttgagcacaaaatttccacggtgaaataaagtcaacattataagctttaaaaaaaaaaa

SEQ ID NO: 10 - protein, 213 aa, Homo sapiens
MRFCPFAERTRLVLKAKGIRHEVININLKNKPEWFFKKNPFGLVPVLENSQGQLIYESAITCEYLDEAYPGKKLLPDDPYEKACQKMILELFSKVPSLVGSFIRSQNKEDYAGLKEEFRKEFTKLEEVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 11 - protein, 241 aa, Homo sapiens
MSGESARSLGKGSAPPGPVPEGSIRIYSMRFCPFAERTRLVLKAKGIRHEVININLKNKPEWFFKKNPFGLVPVLENSQGQLIYESAITCEYLDEAYPGKKLLPDDPYEKACQKMILELFSKVPSLVGSFIRSQNKEDYAGLKEEFRKEFTKLEEVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 12 - protein, 208 aa, Homo sapiens
MSGESARSLGKGSAPPGPVPEGSIRIYSMRFCPFAERTRLVLKAKGIRHEVININLKNKPEWFFKKNPFGLVPVLENSQGQLIYESAITCEYLDEAYPGKKLLPDDPYEKACQKMILELFSKVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 13 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 14 - protein, 183 aa, Homo sapiens
MLSRAVCGTSRQLAPVLGYLGSRQKHSLPDLPYDYGALEPHINAQIMQLHHSKHHAAYVNNLNVTEEKYQEALAKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 15 - protein, 123 aa, Homo sapiens
MSTLSNFTQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGAAGFKMSP

SEQ ID NO: 16 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH